(19) United States
(12) Patent Application Publication
(10) Pub. No.: US 2024/0400992 A1
(43) Pub. Date: Dec. 5, 2024

(54) COMPOSITIONS AND METHODS FOR ANTIGEN-SPECIFIC T CELL EXPANSION

(71) Applicant: MARKER THERAPEUTICS INC., Houston, TX (US)

(72) Inventors: Anastasiya SMITH, Houston, TX (US); Juan F. Vera, Houston, TX (US); Tara Shahim, Houston, TX (US); Eric A. Smith, Houston, TX (US)

(21) Appl. No.: 18/805,940

(22) Filed: Aug. 15, 2024

Related U.S. Application Data
(63) Continuation of application No. PCT/IS2023/062689, filed on Feb. 15, 2023.
(60) Provisional application No. 63/310,500, filed on Feb. 15, 2022.

(51) Int. Cl. C12N 5/0783; C12M 1/04

(52) U.S. Cl. CPC C12N 5/0636; C12M 23/24; C12N 2501/2306; C12N 2501/2307; C12N 2501/2312; C12N 2501/2315; C12N 2501/998

(57) ABSTRACT

The present disclosure provides improved methods for producing tumor antigen-specific T cells, including methods that shorten the procedure as well as enhance certain aspects of the tumor antigen-specific T cells. The present disclosure also provides various compositions and products in connection with the improved production methods. The present disclosure also provides tumor antigen-specific T cell products.

### CROSS-REFERENCE TO RELATED APPLICATION

This application claims priority to U.S. Provisional Application No. 63/310,500, filed Feb. 15, 2022, the content of which is hereby incorporated by reference in its entirety.

### TECHNICAL FIELD

The present disclosure relates to methods for generating tumor antigen-specific T cells, populations, compositions, and products derived thereof, and methods of treatment thereof.

### BACKGROUND

Adoptive transfer of expanded, tumor antigen-specific T cells represents an appealing approach to treating various cancers. The process for generating tumor antigen-specific T cells having the desired activity and phenotype, at the required scale for clinical use, is a challenge. In particular, the length of time required to generate tumor antigen-specific T cells for use in the clinic can be detrimental to patient care. There is a need in the art for improved methods for generating tumor antigen-specific T cells that can be administered to patients for treatment of cancers. There is also a need in the art for improved tumor antigen-specific T cell products which can be used to effectively treat patients. This disclosure addresses this and other needs.

### BRIEF SUMMARY OF CERTAIN EMBODIMENTS OF THE INVENTION

In embodiments, the present disclosure provides methods for generating tumor antigen-specific T cells comprising: (i) placing a population of mononuclear cells obtained from a subject in a vessel comprising at least one gas permeable surface, wherein the mononuclear cells are added to the vessel at a seeding density of about 0.1×10⁶ cells to about 1×10⁷ cells per cm² of the gas permeable surface to form a cell culture; and (ii) adding one or more peptides from one or more tumor antigens to the cell culture, and incubating the one or more peptides with the mononuclear cells in the vessel at about 37° C. in the absence of exogenous cytokines. In some embodiments, the method further comprises adding one or more exogenous cytokines to the cell culture in one embodiment. In a further embodiment, the method comprises harvesting tumor antigen-specific T cells, for example, for autologous transfer or for cryopreservation.

In embodiments, each of the one or more peptides is added to the culture at a concentration of about 0.1 to about 0.5 μg/mL. In embodiments, each of the one or more peptides is added to the culture at a concentration of about 0.1 μg/mL, about 0.2 μg/mL, about 0.25 μg/mL, about 0.3 μg/mL, about 0.4 μg/mL, or about 0.5 μg/mL. In embodiments, the one or more peptides comprise one or more peptides derived from the one or more tumor antigens. In some embodiments, the one or more peptides comprise one or more libraries of peptides derived from the one or more tumor antigens. In embodiments, each library of peptides contains multiple peptides, which in some embodiments span the amino acid sequence of the target tumor antigen. In embodiments, the one or more library of peptides is added to the culture at a concentration of about 0.1 to about 0.5 μg/mL for each peptide. In embodiments, the one or more libraries of peptides is added to the culture at a concentration of about 0.1 μg/mL, about 0.2 μg/mL, about 0.25 μg/mL, about 0.3 μg/mL, about 0.4 μg/mL, or about 0.5 μg/mL for each peptide.

In embodiments, the mononuclear cells and the one or more peptides are incubated in the vessel at about 37° C. prior to addition of the one or more exogenous cytokines. In some embodiments, the mononuclear cells and the one or more peptides are incubated in the vessel for at least about 30 minutes at about 37° C. prior to addition of the one or more exogenous cytokines. In some embodiments, the mononuclear cells and the one or more peptides are incubated in the vessel for at least about 30 minutes to about 30 hours at about 37° C. prior to addition of the one or more exogenous cytokines. In embodiments, the mononuclear cells and the one or more peptides are incubated in the vessel for about 8 to about 24 hours at about 37° C. prior to addition of the one or more exogenous cytokines. In embodiments, the mononuclear cells and the one or more peptides are incubated in the vessel overnight at about 37° C. prior to addition of the one or more exogenous cytokines.

In embodiments, the mononuclear cells are added to the vessel at a seeding density of about 0.1×10⁶ cells to about 1×10⁷ cells per cm² of the gas permeable surface. For example, the mononuclear cells are added to the vessel at a seeding density of about 0.1×10⁶ cells/cm², about 0.5×10⁶ cells/cm², about 1×10⁶ cells/cm², about 2×10⁶ cells/cm², about 3×10⁶ cells/cm², about 4×10⁶ cells/cm², about 5×10⁶ cells/cm², about 6×10⁶ cells/cm², about 7×10⁶ cells/cm², about 8×10⁶ cells/cm², about 9×10⁶ cells/cm² or about 1×10⁷ cells/mL. In embodiments, the mononuclear cells are incubated with the one or more peptides in a cell concentration of about 0.1×10⁶ cells/mL to about 1×10⁷ cells/mL.

For example, the mononuclear cells are added to the vessel at a cell concentration of about 0.1×10⁶ cells/mL, about 0.5×10⁶ cells/mL, about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, or about 1×10⁷ cells/mL. In embodiments, the mononuclear cells are peripheral blood mononuclear cells (PBMCs) or white blood cells.

In embodiments, the mononuclear cells are obtained from a subject (e.g., a donor) via leukapheresis. In embodiments, the donor is a healthy donor. In other embodiments, the donor is the patient to whom the T cells with specificity to multiple tumor-associated antigens (MultiTAA-specific T cells) will be administered after they are generated.

In embodiments, the vessel comprises at least one gas permeable surface. In embodiments, the vessel comprises 1, 2, 3, 4, 5, or 6 gas permeable surfaces. In embodiments, the vessel comprising at least one gas permeable surface is a G-Rex device. For example, in embodiments, the vessel is a 100M G-Rex device or a 500M G-Rex device.

In embodiments, each library of peptides comprises peptides spanning the sequence of the tumor antigen. In embodiments, the method further comprises adding one or more peptides, optionally one or more libraries of peptides, from at least 4 different tumor antigens prior to addition of one or more exogenous cytokines. In embodiments, the method comprises adding one or more peptides, optionally one or more libraries of peptides, from 6 different tumor antigens prior to addition of one or more exogenous cytokines. In embodiments, the method comprises adding one or more peptides, optionally one or more libraries of peptides, from 4, 6, 8, 10, or 12 different tumor antigens prior to addition of one or more exogenous cytokines. In embodiments, the tumor antigens are selected from the group consisting of CEA, MHC, CTLA-4, gp100, mesothelin, PRAME (OIP3) PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, Myc, MYCN, RhoC, TRP-2, GD3, Fucosyl GM1, Mesothelin, PSCA, MAGE A1, MAGE A4, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, Mucin-1, BRAF, CCNA1, and Fos-related antigen 1.

In embodiments, the one or more tumor antigens are selected from PRAME, NY-ESO-1, Survivin, and WT1. In embodiments, the tumor antigens are PRAME, NY-ESO-1, Survivin, and WT1, and the total concentration of peptide added to the vessel is about 78 μg/mL. In further embodiments, the vessel is a 100M G-Rex device, and the total amount of peptide (corresponding to PRAME, NY-ESO-1, Survivin, and WT1 antigens) added to the vessel is about 7.775 mg. In additional embodiments, the vessel is a 500M G-Rex device, and the total amount of peptide added to the vessel (corresponding to PRAME, NY-ESO-1, Survivin, and WT1 antigens) is about 38.875 mg. In embodiments, the methods provided herein comprise generating antigen-specific T cells specific for each of PRAME, NY-ESO-1, Survivin, and WT1.

In embodiments, the one or more tumor antigens are selected from PRAME, NY-ESO-1, Survivin, WT1, MAGEA4, and SSX2. In embodiments, the tumor antigens are PRAME, NY-ESO-1, Survivin, WT1, MAGEA4, and SSX2, and the total concentration of peptide added to the vessel is about 108 μg/mL. In further embodiments, the vessel is a 100M G-Rex device, and the total amount of peptide (corresponding to PRAME, NY-ESO-1, Survivin, WT1, MAGEA4, and SSX2 antigens) added to the vessel is about 10.825 mg. In additional embodiments, the vessel is a 500M G-Rex device, and the total amount of peptide added to the vessel (corresponding to PRAME, NY-ESO-1, Survivin, WT1, NY-ESO-1, and SSX2 antigens) is about 54.125 mg. In embodiments, the methods provided herein comprise generating antigen-specific T cells specific for each of PRAME, NY-ESO-1, Survivin, WT1, NY-ESO-1, and SSX2.

In embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more tumor antigens are represented in the one or more peptides incubated with the mononuclear cells. In embodiments, the tumor antigen-specific T cell products produced by the methods herein exhibit antigen specificity for at least 50%, at least 60%, at least 70%, at least 75%, at least 80%, at least 85%, at least 90%, or at least 95% of the tumor antigens represented by the one or more peptides used in the initial culture. In embodiments, the tumor antigen-specific T cell products produced by the methods herein exhibit antigen specificity for each of the tumor antigens represented by the one or more peptides. In embodiments, increasing the number of tumor antigens incubated with the mononuclear cells surprisingly increases the antigen specificity as well as antigen diversity of the tumor antigen-specific T cell products produced.

In embodiments, the mononuclear cells are obtained from the subject by leukapheresis. The subject may be a healthy donor or may be a patient who has been diagnosed with cancer. In embodiments, the mononuclear cells are white blood cells or peripheral blood mononuclear cells (PBMCs). In embodiments, the method for generating tumor antigen-specific T cells provided herein comprises depleting CD56+ cells from the population of mononuclear cells obtained from the subject. In embodiments, the depletion is carried out by one or more conventional methods such as (but not limited to) magnetic activated cell sorting (MACS), fluorescence activated cell sorting (FACS), Dynabeads, EasySep™ Cell Separation beads, RosetteSep™ Immunodensity Cell Separation, or the Cloudz Human Cell Expansion platform. In embodiments, the population of mononuclear cells placed into the vessel for incubation with the one or more peptides comprises between about 0.05% to about 2.5% CD56+ cells. In embodiments, the population of mononuclear cells placed into the vessel for incubation with the one or more peptides comprises no more than about 5%, no more than about 2.5%, or no more than about 2% CD56+ cells. In embodiments, the population of mononuclear cells placed into the vessel for incubation with the one or more peptides comprises about 35% to about 85% T cells. In embodiments, the population of T cells comprises both CD4+ and CD8+ T cells.

In embodiments, the method for generating tumor antigen-specific T cells provided herein comprises depleting CD19+ cells from the population of mononuclear cells obtained from the subject. In embodiments, the depletion of CD19+ cells is carried out by one or more conventional methods such as (but not limited to) magnetic activated cell sorting (MACS), fluorescence activated cell sorting (FACS), Dynabeads, EasySep™ Cell Separation beads, RosetteSep™ Immunodensity Cell Separation, or the Cloudz Human Cell Expansion platform.

In embodiments, the method for generating tumor antigen-specific T cells provided herein comprises depleting CD56+ cells and CD19+ cells from the population of mononuclear cells obtained from the subject. In embodiments, the depletion of both CD56+ and CD19+ cells is carried out by one or more conventional methods such as (but not limited to) magnetic activated cell sorting (MACS), fluorescence activated cell sorting (FACS), Dynabeads, EasySep™ Cell Separation beads, RosetteSep™ Immunodensity Cell Separation, or the Cloudz Human Cell Expansion platform. In embodiments, the tumor antigen-specific T cell product provided herein generated by a method comprising depletion of CD19+ T cells is suitable for use in treatment of a patient with a B cell cancer.

In embodiments, the population of mononuclear cells placed in the vessel for incubation with the one or more peptides comprises: between about 0.01% to about 2% CD56+CD56+ cells, between about 0.5% to about 50% monocytes, between about 0.01% to about 1.5% CD19+ B cells, and/or between about 40% to about 99% T cells.

In embodiments, the method for generating tumor antigen-specific T cells provided herein comprises adding one or more exogenous cytokines to the culture after the mononuclear cells and peptide have been incubated together in the absence of exogenous cytokine. In embodiments, the method for generating tumor antigen-specific T cells provided herein comprises adding one or more exogenous cytokines to the culture after the mononuclear cells and peptide have been incubated together for at least 8 hours in the absence of exogenous cytokine. In embodiments, the one or more exogenous cytokines added to the culture are selected from IL-6, IL-7, IL-15, IL-12, and any combination thereof. In further embodiments, the exogenous cytokines added to the culture comprise IL-6, IL-7, IL-15, and IL-12.

In embodiments, the mononuclear cells are cultured in the presence of the one or more peptides and the one or more exogenous cytokines for about 5 to about 14 days. Thus, in embodiments, the method for generating tumor antigen-specific T cells provided herein comprises incubating mononuclear cells with one or more peptides in a vessel comprising at least one gas permeable surface for about 8 to about 30 hours at about 37° C. in the absence of exogenous cytokines, and subsequently adding one or more exogenous cytokines to the culture, wherein the mononuclear cells, one or more peptides, and one or more exogenous cytokines are subsequently cultured together in the vessel at about 37° C. for about 5 to about 14 days. In embodiments, the method does not comprise a wash step. In embodiments, the mononuclear cells, one or more peptides, and exogenous cytokines are cultured for about 5 to about 14 days without disruption, for example, without interventions such as adding media or media exchange, adding additional peptide, adding additional cytokines, or adding any other components.

In some embodiments, the method comprises dividing the culture following incubation of the mononuclear cells with the one or more peptides and prior to addition of one or more exogenous cytokines, into two or more individual cultures. For example, in some embodiments, dividing the initial culture into two or more cultures allows for further expansion of the tumor antigen-specific T cells in the culture in the presence of the one or more exogenous cytokines.

In embodiments, the methods further comprise cryopreserving the mononuclear cells and then thawing the cryopreserved cells prior to incubation with the one or more peptides. In embodiments, the thawed mononuclear cells are rested overnight prior to incubation with the one or more peptides. In embodiments, the thawed mononuclear cells are rested for about 4 to about 48 hours prior to incubation with the one or more peptides. In embodiments, the method further comprises counting the cells remaining after the resting period, prior to initiating the culture with the one or more peptides. In embodiments, in this way the seeding density for the culture of the mononuclear cells with the one or more peptides will be accurately calculated, e.g., for placing in the vessel mononuclear cells at a seeding density of about 0.1×10⁶ cells/cm² to about 1×10⁷ cells/cm² (e.g., about 2×10⁶ cells/cm² or about 3×10⁶ cells/cm² or about 4×10⁶ cells/cm²) and/or at a concentration of about 0.1×10⁶ cells/mL to about 1×10⁷ cells/mL (e.g., about 1×10⁶ cells/mL or about 2×10⁶ cells/mL or about 3×10⁶ cells/mL).

In embodiments, the tumor antigen-specific T cells produced by the methods provided herein are cryopreserved after harvest. In embodiments, the mononuclear cells and/or the tumor antigen-specific T cells are cryopreserved in CryoStor® CS10 or a derivative thereof.

In embodiments, the methods provided herein further comprise HLA typing a donor from which the population of mononuclear cells is obtained. In embodiments, the methods provided herein comprise storing the HLA typing information regarding a donor and maintaining a bank of one or more tumor antigen-specific T cell products which can be used as an off the shelf product. In embodiments, the off the shelf product is suitable for use in patients having a desired level of HLA matching with the donor of a tumor antigen-specific T cell product, for example, at least one HLA match.

In embodiments, the method for generating tumor antigen-specific T cells provided herein comprises harvesting the tumor antigen-specific T cells after incubation with first one or more peptides and then one or more exogenous cytokines. In some embodiments, harvesting the tumor antigen-specific T cells does not comprise purification or enrichment based on antigen specificity. In a further embodiment, the method comprises harvesting tumor antigen-specific T cells for cryopreservation and/or storage, for example as an off-the-shelf product, optionally as part of a cell bank or inventory.

In embodiments, the present disclosure provides a population of tumor antigen-specific T cells obtained by the methods provided herein. In embodiments, the present disclosure provides a composition comprising a population of tumor antigen-specific T cells obtained by the methods provided herein. In embodiments, the population of tumor antigen-specific T cells exhibits a superior antigen specificity compared to tumor antigen-specific T cells produced by prior methods. In embodiments, the magnitude of specificity of the tumor antigen-specific T cells for the tumor antigen is at least about 500 SFU/2×10⁵ cells, at least about 700 SFU/2×10⁵ cells, at least about 1,000 SFU/2×10⁵ cells, or at least about 1,200 SFU/2×10⁵ cells as measured by IFN-γ ELISpot.

In embodiments, the present disclosure provides a population of antigen-specific T cells, comprising between about 80% to about 99% CD3+ T cells, <1% CD14+ monocytes, <10% CD19+ B cells, and <10% CD3-CD56+NK cells, wherein the population of antigen-specific T cells has activity against one or more peptides of one or more antigens that the population of T cells has not been previously incubated with. In embodiments, the population of antigen-specific T cells comprises a magnitude of specificity against the at least one peptide of at least one antigen that the population of T cells has not been previously incubated with is, cumulatively, at least about 100 SFU/2×10¹ cells as measured by IFN-γ ELISpot. In embodiments, the magnitude of specificity against at least one antigen that the population has not been previously incubated with is at least about 30 SFU/2×10¹ cells as measured by IFN-γ ELISpot. In embodiments, the population has previously been incubated with one or more peptides from one or more tumor antigens in the absence of exogenous cytokines.

In embodiments, the tumor antigen-specific T cells comprise at least about 20% central memory T cells (T_{CM}). In embodiments, the tumor antigen-specific T cells comprise about 40% effector memory T cells (T_{EM}). In embodiments, the tumor antigen-specific T cells comprise no more than 10% terminally differentiated effector memory cells expressing CD45RA (T_{EMRA}). In embodiments, the tumor antigen-specific T cells comprise at least about 20% naïve T cells. In embodiments, tumor antigen-specific T cells in the product exhibit activity against at least 75% of the tumor antigens represented by the one or more peptides. In embodiments, the tumor antigen-specific T cells in the product exhibit activity against each tumor antigen represented by the one or more peptides. In embodiments, the activity against the antigens, or antigen specificity, is detected by IFNγ production. In embodiments, the IFNγ production is determined by IFNγ ELISpot assay.

In some embodiments, an increased number of tumor antigens represented by the one or more peptides, optionally libraries of peptides, results in (a) an increase in specificity of the antigen specific T cells and/or (b) an increase in specificity of the antigen-specific T cells for the tumor antigens.

In some embodiments, T cells present within said population exhibit activity against one or more peptides of one or more antigens that the population has not been previously incubated with (e.g., “non-target” antigens). In some embodiments, said activity against the one or more peptides of one or more antigens that the population has not been previously incubated with is a result of epitope spreading.

In some embodiments, the magnitude of specificity against the one or more peptides of one or more antigens that the population has not been previously incubated with is, cumulatively, at least about 100 SFU/2×10⁵ cells as measured by IFN-γ ELISpot. In some embodiments, the magnitude of specificity against at least one antigen that the population has not been previously incubated with is at least about 30 SFU/2×10⁵ cells as measured by IFN-γ ELISpot.

In embodiments, the population of antigen-specific T cells has not undergone a purification or enrichment based on antigen specificity.

In embodiments, the present disclosure provides a composition comprising a population of ex vivo expanded tumor antigen-specific T cells comprising CD4+ T cells and CD8+ T cells and comprising a CD56+NK cell component of less than about 10%, wherein the magnitude of specificity for the tumor antigens is at least about 500 SFU/2×10⁵ cells as measured by IFN-γ ELISpot. In some embodiments, said magnitude of specificity for the tumor antigens is at least about 700 SFU/2×10⁵ cells as measured by IFN-γ ELISpot. In embodiments, the composition additionally comprises a pharmaceutically acceptable buffer or excipient. In further embodiments, the composition comprises a CD19+ component of less than about 10%. In embodiments, both the CD4+ and CD8+ T cell compartments exhibit specificity for the tumor antigens.

In embodiments, the tumor antigen-specific T cells are specific for PRAME, NY-ESO-1, Survivin, and WT-1. In embodiments, the tumor antigen-specific T cells are specific for PRAME, NY-ESO-1, Survivin, WT-1, SSX2, and MAGEA4. In embodiments, the tumor antigen-specific T cells are specific for PRAME, NY-ESO-1, Survivin, WT1, SSX2, and/or MAGEA4, and are specific for 1, 2, 3, 4, 5, 6, or more additional tumor associated antigens. In embodiments, the tumor antigen-specific T cells comprise at least about 20% central memory T cells (T_{CM}). In embodiments, the tumor antigen-specific T cells comprise at least about 20% naïve T cells. In embodiments, the tumor antigen-specific T cells comprise about 40% effector memory T cells (T_{EM}). In embodiments, the tumor antigen-specific T cells comprise no more than 10% terminally differentiated effector memory cells expressing CD45RA (TEMRA).

In an aspect, the present disclosure provides a method for generating tumor antigen-specific T cells, comprising:
(i) placing a population of white blood cells at a density of 0.1-9×10⁶ cells/cm² in a static cell culture device with at least one gas permeable surface, with one or more peptides of one or more tumor associated antigens, wherein each peptide is present in the cell culture device at a concentration of about 0.1 μg/mL to about 0.5 μg/mL, and wherein the white blood cells comprise about 1% to about 50% monocytes and about 40% to about 99% T cells;
(ii) incubating the cells and peptides of step (i) at about 37° C. and 5% CO₂ for about 8 to about 30 hours;
(iii) adding one or more exogenous cytokines after the about 8 to about 30 hour incubation;
(iv) culturing the cells, peptides, and exogenous cytokines for about 5 to about 14 days, wherein the culture is not disturbed after addition of the exogenous cytokines.

In embodiments, the method additionally comprises, after the culture of (iv), harvesting the tumor antigen-specific T cells. In embodiments, the one or more peptides are from four tumor associated antigens, and the total concentration of peptide present in the cell culture device at step (i) is about 78 μg/mL. In embodiments, the one or more peptides are from six tumor associated antigens, and the total concentration of peptide present in the cell culture device at step (i) is about 108 μg/mL. In embodiments, the method does not comprise purification or enrichment based on antigen specificity of the T cells.

In an aspect, the present disclosure provides a T cell product generated by the method recited above. In embodiments, the present disclosure provides a tumor antigen-specific T cell product comprising: about 80% to about 99% CD3+ T cells, <1% CD14+ monocytes, <10% CD19+ B cells, and <10% CD3-CD56+NK cells and a CD3+ T cell compartment comprising about 12% to about 80% CD4+ T cells and about 17% to about 84% CD8+ T cells, wherein the CD3+T compartment comprises of at least about 20% central memory T cells (T_{CM}). In embodiments, the tumor antigen-specific T cells comprise at least about 20% naïve T cells. In embodiments, the tumor antigen-specific T cells comprise about 40% effector memory T cells (T_{EM}). In embodiments, the tumor antigen-specific T cells comprise no more than 10% terminally differentiated effector memory cells expressing CD45RA (TEMRA).

In embodiments, the T cell product comprises cells capable of producing IFN-γ as determined by enzyme-linked immune absorbent spot (ELISpot) assay, for example, wherein the magnitude of specificity of the tumor antigen-specific T cells for the tumor antigens is at least about 500 SFU/2×10⁵ cells for the sum of all antigens. In embodiments, the magnitude of specificity of the antigen-specific T cell product against antigens not represented by the one or more peptides is, cumulatively, at least about 100 SFU/2×10⁵ cells. In embodiments, the magnitude of specificity of the antigen-specific T cell product against antigens not represented by the one or more peptides is, cumulatively, at least about 30 SFU/2×10⁵ cells. In embodiments, the T cell product is cryopreserved. In embodiments, the T cell product is suitable for use in an autologous cell therapy for treatment of a cancer and/or for use in an allogeneic cell therapy for treatment of a cancer.

In embodiments, the present disclosure provides methods for treating a patient comprising administering to the patient a composition and/or population of antigen-specific T cells provided herein. The population of antigen-specific T cells are as defined herein, and/or a population of antigen-specific T cells generated by the methods provided herein. In embodiments, the patient has a cancer. In embodiments, the cancer is a leukemia, a lymphoma, or a solid tumor. In embodiments, the cancer is selected from the group consisting of acute myeloid leukemia (AML), myelodysplastic syndrome (MDS), acute lymphoblastic leukemia (ALL), multiple myeloma, non-Hodgkin's lymphoma, sarcoma, breast cancer, and pancreatic cancer. In embodiments, the patient has undergone an allogeneic hematopoietic stem-cell transplant (HSCT). In some embodiments, the patient is immunocompromised. In other embodiments, the patient is not immunocompromised. In some embodiments, the patient is the same individual as the subject from which the mononuclear cells were obtained, that is, the method of treatment is autologous. In other embodiments, the patient is a different individual, that is, the method of treatment is allogeneic. In some embodiments, the patient is partially HLA matched to the subject from which the mononuclear cells were obtained. In some embodiments, the patient has at least one HLA match to the subject from which the mononuclear cells were obtained. In some embodiments, the methods comprise HLA typing the patient prior to treatment. In some embodiments, the methods comprise selecting a tumor antigen-specific T cell product from a bank of products based on the HLA type of the product and the patient.

In embodiments, the methods of treatment provided herein further comprise administering one or more additional populations (e.g., a second population, optionally a third population, optionally a fourth population, optionally a fifth population, and optionally a sixth population) of antigen-specific T cells to the patient, wherein the one or more additional populations of antigen-specific T cells are antigen-specific T cells or antigen-specific T cell products as described herein and/or are obtained by the method provided herein, and wherein the method comprises treating the patient with the one or more additional populations of antigen-specific T cells at least about 2, about 3, about 4, about 5, or about 6 weeks after treatment with the previously-administered population. In embodiments, the one or more additional populations (e.g., second and/or third populations) of antigen-specific T cells, for autologous treatments, may be generated after obtaining one or more additional (e.g., a second, and/or a third) populations of mononuclear cells from the patient at least about 3, about 4, about 5, about 6, about 7, or about 8 weeks after the previous (e.g., first and/or second) population was administered to the patient. In embodiments, said one or more additional populations may contain an amplified number of antigen-specific cells compared to the previous (e.g., first) population, and may comprise a more potent dose. In embodiments, the one or more additional populations of antigen-specific T cells, for an allogeneic treatment, are obtained from a bank of cryopreserved products, or are generated after obtaining one or more additional populations of mononuclear cells from a different subject than the patient (e.g., a donor).

In embodiments, the methods provided herein further comprise comprising monitoring disease progression in the patient by measuring RNA expression of one or more of the tumor antigens. In embodiments, the expression of the one or more tumor antigens correlates with minimal residual disease (MRD) in the patient. In embodiments, the methods further comprise measuring RNA expression of one or more additional tumor antigens that are not tumor antigens corresponding to the one or more peptides. In embodiments, the one or more additional tumor antigens corresponds with MRD in the patient. In embodiments, the methods provided herein result in epitope spreading to additional tumor antigens that are not tumor antigens corresponding to the one or more peptides used to generate the antigen-specific T cell product (see, e.g., FIGS. 24A-24G).

In embodiments, the present disclosure provides cell compositions as starting material for generating the antigen-specific T cell products provided herein. In embodiments, the compositions comprise mononuclear cells which have been depleted of CD56+ cells. In further embodiments, the composition comprises between about 40% to about 99% T cells and no more than about 2% NK cells. In embodiments, the composition further comprises one or more peptides from one or more tumor antigens. In embodiments, the composition further comprises one or more libraries of peptides from one or more tumor antigens. In further embodiments, each library of peptides comprises peptides spanning the sequence of a tumor antigen. Thus, each library of peptides comprises multiple different peptides, each corresponding to all or a portion of the tumor antigen. In embodiments, each peptide in the composition is present at a concentration of about 0.1 μg/mL to about 0.5 μg/mL, for example about 0.2 μg/mL, about 0.25 μg/mL, about 0.3 μg/mL, about 0.4 μg/mL, or about 0.5 μg/mL. In embodiments, the composition comprises peptides, optionally libraries of peptides, from at least 4 different tumor antigens, e.g., from 4, 5, 6, 7, 8, 9, 10, 11, or 12 different tumor antigens. In embodiments, the tumor antigens are selected from PRAME, NY-ESO-1, Survivin, WT1, SSX2, and MAGEA4. In embodiments, the tumor antigens are PRAME, NY-ESO-1, Survivin, and WT1. In embodiments, the composition is present in a vessel comprising at least one gas permeable surface, e.g., comprising 1, 2, 3, 4, 5, or 6 gas permeable surfaces. In embodiments, the composition is in a vessel and the mononuclear cells are present at a seeding density of about 0.1×10⁶ cells to about 9×10⁶ cells per cm² of the gas permeable surface.

### BRIEF DESCRIPTION OF THE DRAWINGS

FIG. 1 provides a schematic of one embodiment of the method for generating tumor antigen-specific T cells provided herein. The procedure shown in FIG. 1A is referred to herein as “MAT110-CD56+Depl”. FIGS. 1B and 1C provide schematics of two prior methods for generating tumor antigen-specific T cells. FIG. 1B provides the method referred to herein as “MAT101-4” which involves stimulation of PBMCs with cytokines to generate dendritic cells (DC) which are subsequently pulsed with a peptide mix and incubated with cytokines, including two or more media exchange steps and peptide pulse steps, for a total culture period of about 31-36 days. FIG. 1C provides the method referred to herein as “MAT103-4,” in which CD14+ PBMCs are cultured in the presence of cytokines to generate mature DCs, which are then pulsed with peptide and incubated with CD56− cells and cytokines and cultured for a total of 15-20 days.

FIGS. 2A, 2B, and 2C provide the details of starting material for generation of tumor antigen-specific T cells. FIG. 2A shows the components of PBMC starting material that was not subject to depletion of any cell type. FIG. 2B shows the components of PBMC starting material that was depleted of CD56+ cells. FIG. 2C shows the components of PBMC starting material that was depleted of CD56+ cells and CD19+ cells.

FIG. 3 provides the details of the tumor antigen-specific T cell product prepared using non-depleted starting material according to method “MAT110-NoDepl”. The % of viable cells that are monocytes, T cells, B cells, and NK cells are shown in the first panel. The % of CD3+ T cells in the final product that are CD4+ or CD8+ is shown in the second panel. The phenotype (Tₙₐᵢᵥₑ, T_{CM}, T_{EM}, and T_{EMRA}) of the CD3+ T cells in the final product are shown in the third panel. The fold expansion of cells in the final product is shown in the fourth panel. The combined antigen specificity (SFU/2×10⁵ cells, as measured by IFNγ ELISpot) is shown in the fifth panel. Statistical analyses are shown in the tables below each panel. SFU=Spot Forming Units.

FIG. 4 provides the details of the tumor antigen-specific T cell product prepared using CD56+ depleted cells as starting material according to method “MAT110-CD56+Depl”. The % of viable cells that are monocytes, T cells, B cells, and NK cells are shown in the first panel. The % of CD3+ T cells in the final product that are CD4+ or CD8+ is shown in the second panel. The phenotype (Tₙₐᵢᵥₑ, T_{CM}, T_{EM}, and T_{EMRA}) of the CD3+ T cells in the final product are shown in the third panel. The fold expansion of cells in the final product is shown in the fourth panel. The combined antigen specificity (SFU/2×10⁵ cells, as measured by IFNγ ELISpot) is shown in the fifth panel. Statistical analyses are shown in the tables below each panel. SFU=Spot Forming Units.

FIG. 5 provides the details of the tumor antigen-specific T cell product using CD56+ and CD19+ depleted cells as starting material. The procedure used to generate these tumor antigen-specific T cells is referred to as “MAT210” and is identical to the “MAT110-CD56+Depl” procedure shown in FIG. 1A except that the mononuclear cells are depleted of CD19+ cells as well as CD56+ cells prior to the peptide pulse. The % of viable cells that are monocytes, T cells, B cells, and NK cells are shown in the first panel. The % of CD3+ T cells in the final product that are CD4+ or CD8+ is shown in the second panel. The phenotype (Tₙₐᵢᵥₑ, T_{CM}, T_{EM}, and T_{EMRA}) of the CD3+ T cells in the final product are shown in the third panel. The fold expansion of cells in the final product is shown in the fourth panel. The combined antigen specificity (SFU/2×10⁵ cells, as measured by IFNγ ELISpot) is shown in the fifth panel. Statistical analyses are shown in the tables below each panel. SFU=Spot Forming Units.

FIGS. 6A and 6B show a comparison between the improved method provided herein (referred to as “MAT 110-CD56+Depl” and shown schematically in FIG. 1A), MAT110-No Depl, and prior method MAT103-4 (shown schematically in FIG. 1C). Addition of the CD56+ depletion step significantly improved the fold expansion of T cells generated using MAT110 method.

FIGS. 7A and 7B show the phenotype of T cells generated using the MAT110-CD56+Depl method shown schematically in FIG. 1A) compared to MAT103-4 obtained using the prior method shown schematically in FIG. 1C. The MAT110-CD56+Depl method resulted in products with comparable distribution of CD4+ and CD8+ T cells and a greater proportion of central memory T cells (T_{CM}).

FIGS. 8A and 8B show the antigen specificity of MAT110-CD56+Depl cells obtained using the improved method provided herein compared to MAT103-4 and MAT110-No-Depl cells obtained via the prior methods, respectively. FIG. 8A shows the antigen specificity in SFU/2×10⁵ cells as measured by IFNγ ELISpot, for cells from individual donors. Each panel provides the results for cells obtained from an individual donor. FIG. 8B provides the total magnitude of antigen specificity (combined reactivity against each target antigen, when donor data was pooled)), and shows that the increase in the magnitude of response that was achieved with the MAT110-CD56+Depl method was statistically significant. SFU=Spot Forming Units.

FIG. 9A shows the total magnitude of response (sum of all antigens) in SFU/2×10⁵ cells as measured by IFNγ ELISpot. Products generated using MAT110-CD56 Depl had a larger number of antigen-specific T cells. FIG. 9B shows the spectrum of antigen specificity of the tumor antigen-specific T cell products generated by the prior methods versus MAT110-CD56+Depl. The MAT110-CD56 Depl manufacturing process increased the proportion of multi-targeted T cells. FIG. 9C shows the increased number of products with specificity for multiple tumor antigens that is achieved with the MAT110-CD56+Depl cells. “Run” refers to a manufacturing run that generates a T-cell product using cells FIG. 9D shows a linear correlation between % the anti-tumor activity (as measured in in vitro coculture experiments) and antigen specificity (as measured by IFNγ ELISpot, SFU/2×10⁵ cells, x axis) of T cell products. Additionally, FIG. 9D demonstrates that T cell products generated using the MAT110-CD56+Depl method have higher antigen specificity and anti-tumor activity compared to those generated using the MAT103-4 method.

FIG. 10 provides exemplary calculations for peptide amounts for the four-antigen pool (PRAME, NY-ESO-1, Survivin, and WT1) and 6 antigen pool (PRAME, NY-ESO-1, Survivin, WT1, MAGEA4, and SSX2), and total peptide amounts used in a 100M G-Rex or a 500M G-Rex device.

FIG. 11 provides the antigen specificity of MultiTAA-specific T cell products generated by performing the initial peptide pulse step in the presence of cytokines (+cytokines) versus performing the initial peptide pulse step in the absence of cytokines (-cytokines). PBMCs from healthy donors were pulsed with peptides from six different antigens (WT1, Survivin, NY-ESO-1, PRAME, SSX2 and MAGEA4) overnight, either in the presence or absence of cytokines (IL-6, IL-12, IL-7 and IL-15) for the manufacture of MultiTAA-specific T cells. MultiTAA-specific T cells were subjected to IFN-γ ELISpot analysis as a readout of antigen specificity. SFU=Spot Forming Units.

FIG. 12 provides the antigen specificity of MultiTAA-specific T cell products generated by using a high versus low volume for the overnight peptide pulse step. PBMCs from healthy donors were pulsed with peptides from 4 different antigens (WT1, Survivin, NY-ESO-1, and PRAME) overnight, either in a low volume (10 mL) or high volume (100 mL) for the manufacture of MultiTAA-specific T cells (n=3). MultiTAA-specific T cells were subjected to IFN-γ ELISpot analysis as a readout of antigen specificity. SFU=Spot Forming Units.

FIG. 13 shows the fold expansion (bar graph, left), % viability (line graph, above), and antigen specificity (bar graph, right) of MultiTAA-specific T cell products generated using a positively selected vs negatively selected cell population as a starting material for the culture. PBMCs were either positively selected for T cells and monocytes (CD8+CD4+CD14+) prior to placing the cells in the culture, or depleted of CD56+NK cells (CD56^{pos} Depl) prior to placing the cells in the culture. MultiTAA-specific T cells were subjected to IFN-γ ELISpot analysis as a readout of antigen specificity.

FIG. 14 shows a correlation between T cell specificity for target antigens and minimal residual disease (MRD). % MRD (left y-axis and solid line) and antigen specificity (right y-axis and bars) were determined pre-infusion and at week 8, 12, 18, 24, and 32 following administration of MT101-4 cells. Target antigen-specific T cells were present in patient peripheral blood and inversely correlated with MRD, suggestive of anti-tumor activity.

FIG. 15 shows a correlation between T cell specificity for non-target antigens (bars) and MRD (solid line). T cells specific for the indicated additional antigens (i.e., “non-target antigens” that were not used to generate the antigen-specific T cell product) that were present in patient peripheral blood after treatment inversely correlated with MRD, representing epitope spreading.

FIG. 16 shows T cell gene expression in patient bone marrow pre-infusion and at weeks 8, 12, 18, 24, and 32 after treatment. MRD is also shown. Gene expression was determined by next generation sequencing (NGS).

FIG. 17 shows the T cell composition and phenotype in the patient pre-infusion and at weeks 8, 12, and 24 after treatment. CD4 and CD8 populations are shown in the top row. The bottom row shows CD197 and C45RA staining, and the naïve (CD197+CD45RA+), central memory (T_{CM}, CD197+CD45RA−), effector memory (T_{EM}, CD197-CD45RA−) and terminally differentiated (T_{EMRA}, CD197-CD45RA+) populations are indicated.

FIG. 18 shows that both CD4+ and CD8+ T cells in the patient are functional (express IFNγ, as shown in the bars), and function correlates with MRD (solid line).

FIG. 19 shows IFNγ expression by Lag3− and Lag3+ T cells in the patient after infusion.

FIG. 20 shows expansion of peripheral T cell clones in the patient after infusion.

FIG. 21 shows target antigen RNA expression in the bone marrow of the patient pre-infusion and at weeks 8, 12, 18, 24, and 32. The top dotted line represents RNA expression of Survivin; the bottom dotted line represents RNA expression of WT1. The solid line is % MRD. Bars show specificity for the indicated antigens. *=PRAME and NY-ESO-1 transcripts were not detected.

FIG. 22 shows RNA expression of non-target antigen proteinase-3 (dotted line) in the bone marrow of the patient pre-infusion and at weeks 8, 12, 18, 24, and 32. The solid line is % MRD. Specificity of patient cells for non-target antigens including proteinase-3 (bars) is also shown for each timepoint.

FIG. 23 shows RNA expression of additional non-target antigens (dotted lines; at week 18, top to bottom are BRAF, TERT, Mucin-1, Myc and CCNA1) in the bone marrow of the patient pre-infusion and at weeks 8, 12, 18, 24, and 32. The solid line is % MRD. Specificity of patient cells for non-target antigens is also shown (bars).

FIG. 24A-G shows that infusion of MultiTAA-specific T cells results in immune responses to additional “non-target” TAAs, representing epitope spreading. FIG. 24A is a schematic of the kinetics of immune response. FIG. 24B shows complete response (CR) in a pancreatic cancer patient after MultiTAA-specific T cell treatment, correlating with epitope spreading as shown in FIG. 24D (bars in the Peak post column, from top to bottom, are: WT1, AFP, MART1, MC1, MA3 [largest portion of bar], MA2B, MA1). Antigen specificity for the infused product for the patient is shown in FIG. 24C (bars in the Peak post column, from top to bottom, are: Survivin, NYESO1 [largest portion of bar], MAGEA4, SSX2, and Prame). FIG. 24E shows partial response (PR) in a pancreatic cancer patient after MultiTAA-specific T cell treatment, correlating with epitope spreading in the patient as shown in FIG. 24G (bars in the Peak post column, from top to bottom, are: WT1, AFP, MART1, MC1, MA3 [largest portion of bar], MA2B, MA1). Antigen specificity for the infused product for the patient is shown in FIG. 24F (bars in the Peak post column, from top to bottom, are: Survivin, NYESO1, MAGEA4, SSX2 [largest portion of bar], and Prame).

FIG. 25A shows imaging of tumor size in a patient administered T cells manufactured using the MAT101 method. Images show tumor pre-infusion and 9 months post infusion with the therapy. FIG. 25B shows antigen specificity as measured by IFN-γ ELISpot analysis for target and non-target antigens at month 3 and month 9 post administered of cells to patients compared to a patient's antigen specificity prior to infusion (bars in the Mth3 and Mth9 columns, from top to bottom, are: Survivin, NYESO1, MAGEA4, SSX2, and Prame).

FIG. 26 shows a graph measuring T-cell expansion in responding and non-responding lymphoma patients administered MultiTAA-specific T cells (a T-cell population generated using a peptides from 5 tumor-associated antigens [TAAs]). Expansion of T cells specific to “target” epitopes from the peptides used to generate the antigen-specific T cell product (left) and “non-target” epitopes indicative of epitopes spreading (right) were measured 6 weeks after the initial infusion of MultiTAA-specific T cells (FIG. 26).

FIGS. 27A-27D show a schematic of the in vitro experimental assay evaluating epitope spreading (FIG. 27A), and associated cell growth (FIG. 27B) and antigen specificity (FIGS. 27C-27D). FIG. 27B shows graphical (left) and visual representation (right, bioluminescence is indicative of cell density) of cell growth of PANC1 cells (a pancreatic ancer cell line) cultured in vitro alone or with non-specific T cells activated with anti-CD3/CD28 antibody or MT-601 antigen specific cells. Antigen specificity was measured for target (FIG. 27C) and non-target antigens (FIG. 27D) by IFN-γ ELISpot analysis.

FIG. 28 is a graph showing the antigen specificity of the MT-401 cell population (a T-cell population generated using peptides from four TAAs), as measured by IFN-γ ELISpot analysis.

FIG. 29 is a schematic demonstrating culture of bone marrow mononuclear cells (BM-MNCs, target cells) with control PBMC's or MT-401 cells (effectors) and analysis of target cell killing in culture, as measured by flow cytometry using the Tmem5 panel.

FIGS. 30A-30B provide graphs measuring glucose (FIG. 30A) and lactate (FIG. 30B) production on days 0, 3, and 4 of co-culture between target cells alone (AML from patient), target cells with PBMCs, or target cells with MT-401.

FIGS. 31A-31B are graphs showing target cell killing (measured by fold change in growth after 4-days of culture) at an effector:target (E:T) ratio of 4:1, using cells derived from a single patient (PT117-201). Changes in cell growth were measured for target cells (BM-MNCs) only, target cells cultured with control PBMCs, and target cells cultured with MT-401. ****=p<0001; ns=not significant.

FIG. 32 is a graph showing target antigen-specificity of MT-401 cells (derived from a single patient 0117-201 and prepared using peptides from four TAAs) prior to culture with target cells (BM-MNCs) and after co-culture with target cells, as measured by IFN-γ ELISpot analysis.

FIGS. 33A-33B show graphs comparing antigen-specificity between treatment groups of target cells (BM-MNCs) alone, target cells with PBMCs, or target cells cultured with MT-401 cells over a 4-day period. Target antigen specificity was observed in the MT-401 group (FIG. 33A) (bars in the Targets+ PBMCs column, from top to bottom, are: WT1, Survivin [largest portion of bar], NY-ESO-1, and Prame; bars in the Targets+MT-401 column, from top to bottom, are: WT1, Survivin, NY-ESO-1, and Prame [largest portion of bar],) and additional non-target antigen specificity was observed in the MT-401 treatment group after co-culture with target cells (FIG. 33B) (bars in the Targets+ PBMCs column, from top to bottom, are: WT1, Survivin [largest portion of bar], NY-ESO-1, and Prame; bars in the Targets+MT-401 column, from top to bottom, are: BRAF, Mucin-1, TERT, Myc, MAGEA4, SXX2, WT1, Survivin, NY-ESO-1, and Prame [largest portion of bar], with CCNA1 and Proteinase-3 not visible).

FIG. 34 is a schematic representation of the off-the-shelf program for donor selection, product manufacture, cell banking, and patient treatment.

FIG. 35 shows the projected number of cell lines needed to reach 90% coverage of a patient population.

FIG. 36 shows T cell fold expansion, % viability, and total viable cells when the MultiTAA-specific T cell product is prepared in the G-Rex 10M vs the G-Rex-500M.

FIG. 37 shows the sum of antigen specificity and the diversity of antigen specificity in cells obtained from three different donors and generated by stimulating with 4 or 6 peptide libraries. As shown in the fourth panel, cells from a fourth donor were stimulated with 12 peptide libraries corresponding to the 12 indicated tumor antigens (bars in the 4 Ag columns, from top to bottom, are: WT1, Survivin, NY-ESO-1, and PRAME; bars in the 6 Ag columns, from top to bottom, are: MAGEA4, SSX2, WT1, Survivin, NY-ESO-1, and PRAME; bars in the 12 Ag column, from top to bottom, are: Proteinase-3, CCNA1, BRAF, Mucin-1, TERT, Myc, MAGEA4, SSX2, WT1, Survivin, NY-ESO-1, and PRAME).

### DETAILED DESCRIPTION

In one aspect, the present disclosure provides improved methods for generating tumor antigen-specific T cells. The tumor antigen-specific T cells described herein and generated by way of the methods provided herein have superior properties relative to previously described tumor antigen-specific T cells including an improved magnitude of response to targeted tumor antigens, increased specificity, increased viability, and a desirable phenotype that leads to efficient killing of tumor cells upon administration to a patient. In some embodiments, the present disclosure provides methods for generating a tumor antigen-specific T cell product generated from mononuclear cells of a patient or subject (e.g., a donor). In some embodiments, the tumor antigen-specific T cell product is generated via the methods provided herein from mononuclear cells of a patient, and is subsequently administered to the patient (autologous use). In some embodiments, the tumor antigen-specific T cell product is generated via the methods provided herein from mononuclear cells of a donor, and is maintained in a cryopreserved state until use in a patient in need thereof, which may be the donor or may be a different individual (autologous or allogeneic use).

In embodiments, the method provided herein comprises culturing mononuclear cells from a subject in a vessel with a high concentration of peptides, and incubating the cells and the peptides together before adding one or more exogenous cytokines to the culture (also referred to herein as a peptide “pulse”). The present inventors found that surprisingly, the pre-incubation of the cells with the peptides prior to addition of one or more exogenous cytokines significantly improved various aspects of the tumor antigen specific T cells generated by the process, including antigen specificity and phenotype. Given the correlation between the sum of antigen specificity and anti-tumor activity provided herein, the tumor antigen specific T cells of the present disclosure have significantly more potent anti-tumor activity. In addition, the process provided herein allows for a rapid (5-14 days) production process that is beneficial for patient treatment. For example, when administering tumor antigen-specific T cells to a patient in need thereof, a shorter process time is highly desirable to avoid undue delay in treatment. Such a shorter process time is an improvement over prior methods for producing tumor antigen-specific T cells, which included a process time of more than 14 days, for example 20, 30, 36, or more days.

In embodiments, the method provided herein comprises a first incubation step of mononuclear cells (e.g., PBMCs or white blood cells) with a high concentration of one or more peptides in a low volume. In embodiments, the sending density of the cells is about 0.1×10⁶ cells to about 1×10⁷ cells per cm² of the gas permeable surface. In embodiments, the high concentration of peptide is about 0.1 μg/mL to about 0.5 μg/mL (e.g., about 0.1 μg/mL, about 0.2 μg/mL, about 0.25 μg/mL, 0.3 μg/mL, about 0.4 μg/mL, or about 0.5 μg/mL) and/or the peptide is incubated with the cells in a low volume to facilitate interaction of the cells with the peptide. In some embodiments, the method comprises placing the mononuclear cells into a vessel or device, e.g. at a seeding density of about 0.1×10⁶ cells/cm² to about 1×10⁷ cells/cm², then adding the peptide. In other embodiments, the method comprises placing the peptide mix into the vessel or device, then adding the mononuclear cells, e.g. at a seeding density of about 0.1×10⁶ cells/cm² to about 1×10⁷ cells/cm². In some embodiments, the method comprises mixing the mononuclear cells with the peptide mix and then placing the cells and peptides into the vessel or device. In some embodiments, the method comprises mixing a portion of the cells and/or a portion of the peptides together and then placing the mixture into the vessel or device, then adding the remaining cells and/or peptide mix to the vessel or device.

### Mononuclear Cells

In one aspect, the present disclosure provides methods wherein mononuclear cells are obtained from a subject. In some embodiments, the method provided herein comprises obtaining peripheral blood mononuclear cells (PBMCs) from a subject. In some embodiments, the mononuclear cells are obtained from a subject via leukapheresis. In some embodiments, the mononuclear cells are PBMCs or white blood cells. In some embodiments, said subject is a donor. In some embodiments, the donor is a healthy donor. In some embodiments, the donor is the patient to whom the MultiTAA-specific T cells will be administered after they are generated.

In some embodiments, the method provided herein comprises a depletion step prior to placing the population of mononuclear cells into the vessel for incubation with the one or more peptides. In some embodiments, said depletion step comprises depleting CD56+ cells from the population of mononuclear cells. In some embodiments, the mononuclear cells are depleted of CD56+ cells and comprise CD3+ T cells. In some embodiments, the mononuclear cells are depleted of CD56+ cells and comprise CD3+ T cells and monocytes. In some embodiments, the cells have been depleted of CD56+ cells, such that the cells in the vessel are made up of CD3+ T cells and monocytes, with few contaminating CD56+ cells. In embodiments, the method comprising said depletion step is referred to herein as “MAT110-CD56+Depl”. In some embodiments, a T cell generated by this process is referred to herein as “MAT110-CD56+Depl”. In some embodiments, PBMCs are depleted of CD56+ cells using one or more methods known to those of ordinary skill in the art, such as (but not limited to) magnetic activated cell sorting (MACS), fluorescence activated cell sorting (FACS) Dynabeads, EasySep™ Cell Separation beads, RosetteSep™ Immunodensity Cell Separation, or the Cloudz Human Cell Expansion platform.

In some embodiments, the method provided herein further comprises depleting CD19+ cells from the population of mononuclear cells prior to placing the population of mononuclear cells into a vessel or device for incubation with the one or more peptides. In some embodiments, the method comprising depletion of CD19+ T cells is suitable for use in treatment of a patient with a B cell cancer. In some embodiments, the depletion step comprises depleting the mononuclear cells of CD56+ cells and CD19+ cells. In embodiments, the method comprising said depletion of both CD56+ and CD19+ cells is referred to herein as “MAT210” or “MAT210-CD56+Depl-CD19+Depl”. In some embodiments, a T cell generated by the method provided herein including depletion of both CD56+ and CD19+ cells is referred to herein as “MAT210” or “MAT210-CD56+Depl-CD19+Depl”. In some embodiments, PBMCs are depleted of CD19+ cells using methods known to those of ordinary skill in the art, such as (but not limited to) magnetic activated cell sorting (MACS), fluorescence activated cell sorting (FACS), Dynabeads, EasySep™ Cell Separation beads, RosetteSep™ Immunodensity Cell Separation, or the Cloudz Human Cell Expansion platform.

In some embodiments, the mononuclear cells after depletion of CD56+ cells comprise about 0.05% to about 2.5% CD56+ cells. In some embodiments, the mononuclear cells after depletion of CD56+ cells comprise about 35% to about 85% T cells. In some embodiments, the mononuclear cells after depletion of CD56+ cells comprise about 0.01% to about 2% CD56+ cells, about 0.5% to about 50% monocytes, about 0.01% to about 1.5% CD19+B cells, and/or about 40% to about 99% T cells. In some embodiments, the mononuclear cells after depletion of CD56+ cells comprise no more than about 2.5% CD56+ cells. In some embodiments, the mononuclear cells after depletion of CD56+ cells comprise about 1% to about 50% monocytes and about 40% to about 99% T cells.

In some embodiments, the mononuclear cells after depletion of both CD56+ cells and CD19+ cells comprise about 0.01% to about 2% CD56+ cells. In some embodiments, the mononuclear cells after depletion of both CD56+ cells and CD19+ cells comprise about 40% to about 99% T cells. In some embodiments, the mononuclear cells after depletion of both CD56+ cells and CD19+ cells comprise about 0.01% to about 2% CD56+ cells, about 0.5% to about 50% monocytes, about 0.01% to about 1.5% CD19+ B cells, and/or about 40% to about 99% T cells. In some embodiments, the mononuclear cells after depletion of both CD56+ cells and CD19+ cells comprise no more than about 2.5% CD56+ cells. In some embodiments, the mononuclear cells after depletion of both CD56+ cells and CD19+ cells comprise about 0.5% to about 50% monocytes and about 40% to about 99% T cells.

In one aspect, the present disclosure provides methods wherein said depletion step (e.g., depleting CD56+ cells alone, or depleting CD56+ and CD19+ cells) provides a population of mononuclear cells with certain attributes or characteristics (also called a “cell composition” herein). In some embodiments, said cell compositions may provide starting material for generating the antigen-specific T cell products provided herein. In some embodiments, the population of mononuclear cells or cell composition is placed into a vessel or device for incubation with one or more peptides from one or more tumor associated antigens.

In some embodiments, the population of mononuclear cells placed in the vessel for incubation with the one or more peptides comprises between about 0.05% to about 2.5% CD56+ cells. In some embodiments, the population of mononuclear cells placed in the vessel for incubation with peptides comprises about 35% to about 85% T cells or about 40% to about 99% T cells. In some embodiments, the population of mononuclear cells placed in the vessel for incubation with peptides comprises between about 0.01% to about 2% CD56+NK and CD56+ T cells, between about 0.5% to about 50% monocytes, between about 0.01% to about 1.5% CD19+ B cells, and/or between about 40% to about 99% T cells. In some embodiments, the population of mononuclear cells placed in the vessel with peptides comprise no more than about 2.5% CD56+ cells. In some embodiments, the population of mononuclear cells placed in the vessel for incubation with peptides comprises between about 40% to about 99% T cells and no more than about 2% NK cells.

### Peptides

In one aspect, the present disclosure provides methods wherein the population of mononuclear cells (referred to as “the cells” herein) are incubated with one or more peptides from one or more tumor antigens.

As used herein, peptide is meant to refer to short polymers of amino acids linked by peptide bonds. The peptides in the methods presented herein are sufficiently long to comprise one or more linear epitopes, for example are about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids long.

In embodiments, the one or more peptides comprises peptides corresponding to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more tumor-associated antigen (TAA). For example, in embodiments, the one or more peptides comprises peptides corresponding to at least 4, at least 6, at least 8, at least 12, at least 15, at least 20, at least 30, or at least 40 TAA. In embodiments, the one or more peptides comprise one or more libraries of peptides derived from the one or more TAAs. In embodiments, each library of peptides comprises peptides spanning different portions of the TAA. In embodiments, a library of peptides corresponding to a given tumor antigen includes multiple peptides each spanning all or a portion of the amino acid sequence of the antigen. In embodiments, the one or more peptides is incubated with the cells at a concentration of about 0.1 μg/mL to about 0.5 μg/mL, e.g., about 0.1 μg/mL, about 0.2 μg/mL, about 0.25 μg/mL, about 0.3 μg/mL, about 0.4 μg/mL, or about 0.5 μg/mL. In embodiments, the total concentration of peptide present in the cell culture device during the incubation period with the cells in the absence of exogenous cytokines is at least about 60 μg/mL, for example about 78 μg/mL with an exemplary library of peptides representing four different TAA. In embodiments, the total concentration of peptide present in the cell culture device during the incubation period with the cells in the absence of exogenous cytokines is at least about 100 μg/mL, for example about 108 μg/mL with an exemplary library of peptides representing six different TAA.

In embodiments, each peptide in the composition is present at a concentration of about 0.1 μg/mL to about 0.5 μg/mL, for example about 0.1 μg/mL, 0.2 μg/mL, about 0.25 μg/mL, about 0.3 μg/mL, about 0.4 μg/mL, or about 0.5 μg/mL. In some embodiments, each peptide in the composition is present at a concentration of about 0.1 μg/mL to about 0.5 μg/mL. In some embodiments, each peptide in the composition is present at a concentration of about 0.2 μg/mL to about 0.3 μg/mL. In some embodiments, each peptide in the composition is present at a concentration of about 0.1 μg/mL. In some embodiments, each peptide in the composition is present at a concentration of about 0.2 μg/mL. In some embodiments, each peptide in the composition is present at a concentration of about 0.25 μg/mL. In some embodiments, each peptide in the composition is present at a concentration of about 0.3 μg/mL. In some embodiments, each peptide in the composition is present at a concentration of about 0.4 μg/mL. In some embodiments, each peptide in the composition is present at a concentration of about 0.5 μg/mL.

In some embodiments, the concentration of peptide in the cell culture (e.g., the vessel), is about 50 μg/mL to about 100 μg/mL. In some embodiments, the concentration of peptide in the cell culture is about 50 μg/mL. In some embodiments, the concentration of peptide in the cell culture is about 60 μg/mL. In some embodiments, the concentration of peptide in the cell culture is about 70 μg/mL. In some embodiments, the concentration of peptide in the cell culture is about 75 μg/mL. In some embodiments, the concentration of peptide in the cell culture is about 80 μg/mL. In some embodiments, the concentration of peptide in the cell culture is about 85 μg/mL. In some embodiments, the concentration of peptide in the cell culture is about 90 μg/mL. In some embodiments, the concentration of peptide in the cell culture is about 95 μg/mL. In some embodiments, the concentration of peptide in the cell culture is about 100 μg/mL. In some embodiments, the concentration of peptide in the cell culture is about 105 μg/mL. In some embodiments, the concentration of peptide in the cell culture is about 110 μg/mL. In some embodiments, the concentration of peptide in the cell culture is about 78 μg/mL. In some embodiments, the concentration of peptide in the cell culture is about 108 μg/mL.

In some embodiments, the total amount of peptide added to the vessel is about 6 mg to about 60 mg. In some embodiments, the total amount of peptide added to the vessel is about 6 mg. In some embodiments, the total amount of peptide added to the vessel is about 7 mg. In some embodiments, the total amount of peptide added to the vessel is about 8 mg. In some embodiments, the total amount of peptide added to the vessel is about 9 mg. In some embodiments, the total amount of peptide added to the vessel is about 10 mg. In some embodiments, the total amount of peptide added to the vessel is about 11 mg. In some embodiments, the total amount of peptide added to the vessel is about 12 mg. In some embodiments, the total amount of peptide added to the vessel is about 13 mg. In some embodiments, the total amount of peptide added to the vessel is about 14 mg. In some embodiments, the total amount of peptide added to the vessel is about 15 mg. In some embodiments, the total amount of peptide added to the vessel is about 20 mg. In some embodiments, the total amount of peptide added to the vessel is about 25 mg. In some embodiments, the total amount of peptide added to the vessel is about 30 mg. In some embodiments, the total amount of peptide added to the vessel is about 35 mg. In some embodiments, the total amount of peptide added to the vessel is about 40 mg. In some embodiments, the total amount of peptide added to the vessel is about 45 mg. In some embodiments, the total amount of peptide added to the vessel is about 50 mg. In some embodiments, the total amount of peptide added to the vessel is about 55 mg. In some embodiments, the total amount of peptide added to the vessel is about 60 mg. In some embodiments, the total amount of peptide added to the vessel is about 7.775 mg. In some embodiments, the total amount of peptide added to the vessel is about 10.825 mg. In some embodiments, the total amount of peptide added to the vessel is about 38.875 mg. In some embodiments, the total amount of peptide added to the vessel is about 54.125 mg.

In embodiments, the tumor antigens represented in the one or more peptides comprise tumor antigens selected from CEA, MHC, CTLA-4, gp100, mesothelin, PRAME (OIP3) PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, Myc, MYCN, RhoC, TRP-2, GD3, Fucosyl GM1, Mesothelin, PSCA, MAGE A1, MAGEA4, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, Mucin-1, BRAF, CCNA1, Fos-related antigen 1, and any combination thereof. In some embodiments, the tumor antigens represented in the one or more peptides are selected from the group consisting of tumor antigens CEA, MHC, CTLA-4, gp100, mesothelin, PRAME (OIP3) PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, Myc, MYCN, RhoC, TRP-2, GD3, Fucosyl GM1, Mesothelin, PSCA, MAGE A1, MAGEA4, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, Mucin-1, BRAF, CCNA1, Fos-related antigen 1, and any combination thereof.

In some embodiments, the one or more peptides comprise tumor antigen PRAME. In some embodiments, the one or more peptides comprise tumor antigen NY-ESO-1. In some embodiments, the one or more peptides comprise tumor antigen Survivin. In some embodiments, the one or more peptides comprise tumor antigen WT1. In some embodiments, the one or more peptides comprise tumor antigen MAGEA4. In some embodiments, the one or more peptides comprise tumor antigen SSX2. In embodiments, the one or more peptides comprise peptides from tumor antigens PRAME, NY-ESO-1, Survivin, and WT1. In further embodiments, the one or more peptides further comprise peptides from MAGEA4 and SSX2. In some embodiments, the one or more peptides comprise peptides from tumor antigens PRAME, NY-ESO-1, Survivin, WT1, MAGEA4, and SSX2.

In embodiments, the tumor antigens represented in the one or more peptides used to generate the MultiTAA-specific T cells are referred to herein as “target antigens,” “targeted antigens,” “target tumor associated antigens” “target TAAs” and the like. In embodiments, tumor antigens that are not represented in the one or more peptides used to generate the MultiTAA-specific T cells are referred to herein as “non-target antigens” and the like.

### Mononuclear Cell Culture Methods

In one aspect, the present disclosure provides methods wherein a population of mononuclear cells obtained from a subject is placed in a vessel for incubation with one or more peptides. In embodiments, said population is added to the vessel at a seeding density of about 0.1×10⁶ cells to about 9×10⁶ cells per cm² of the gas permeable surface. In embodiments, said population is added to the vessel at a seeding density of about 0.1×10⁶ cells/cm² to about 1×10⁷ cells/cm². In some embodiments, said population is added to the vessel at a seeding density of about 0.1×10⁶ cells/cm². In some embodiments, said population is added to the vessel at a seeding density of about 0.5×10⁶ cells/cm². In some embodiments, said population is added to the vessel at a seeding density of about 1×10⁶ cells/cm². In some embodiments, said population is added to the vessel at a seeding density of about 2×10⁶ cells/cm². In some embodiments, said population is added to the vessel at a seeding density of about 3×10⁶ cells/cm². In some embodiments, said population is added to the vessel at a seeding density of about 4×10⁶ cells/cm². In some embodiments, said population is added to the vessel at a seeding density of about 5×10⁶ cells/cm². In some embodiments, said population is added to the vessel at a seeding density of about 6×10⁶ cells/cm². In some embodiments, said population is added to the vessel at a seeding density of about 7×10⁶ cells/cm². In some embodiments, said population is added to the vessel at a seeding density of about 8×10⁶ cells/cm². In some embodiments, said population is added to the vessel at a seeding density of about 9×10⁶ cells/cm². In some embodiments, said population is added to the vessel at a seeding density of about 1×10⁷ cells/cm².

In embodiments, the population of mononuclear cells is added to a device or vessel comprising at least one gas permeable surface. In embodiments, the population of mononuclear cells is added to a device or vessel comprising at least one gas-permeable surface. In embodiments, the vessel to which the cells are added comprises 1, 2, 3, 4, 5, or 6 gas permeable surfaces. In some embodiments, the vessel comprises 1 gas permeable surface. In some embodiments, the vessel comprises 2 gas permeable surfaces. In some embodiments, the vessel comprises 3 gas permeable surfaces. In some embodiments, the vessel comprises 4 gas permeable surfaces. In some embodiments, the vessel comprises 5 gas permeable surfaces. In some embodiments, the vessel comprises 6 gas permeable surfaces. In some embodiments, the device or vessel comprising at least one gas permeable surface is a G-Rex device (commercially available from Wilson Wolf Manufacturing). In some embodiments, the vessel is a 100M G-Rex device. In some embodiments, the vessel is a 500M G-Rex device. In other embodiments, the device or vessel comprising at least one gas permeable surface is a gas permeable bag. The devices and vessels suitable for use in the methods provided herein may, for example, allow for cell populations to expand by about 10 fold, about 100 fold, about 1000 fold, or more.

In some embodiments, the cells are incubated in said vessel or device with the one or more peptides at about 37° C. and 5% CO₂. In some embodiments, the method comprises incubating the cells with the peptides for about 30 minutes to about 30 hours at about 37° C. and 5% CO₂. In some embodiments, the method comprises incubating the cells with the peptides for about 8 hours to about 24 hours at about 37° C. and 5% CO₂. In some embodiments, the cells are incubated with peptides for about 30 minutes. In some embodiments, the cells are incubated with peptides for about 1 hour. In some embodiments, the cells are incubated with peptides for about 2 hours. In some embodiments, the cells are incubated with peptides for about hours. In some embodiments, the cells are incubated with peptides for about 4 hours. In some embodiments, the cells are incubated with peptides for about 5 hours. In some embodiments, the cells are incubated with peptides for about 6 hours. In some embodiments, the cells are incubated with peptides for about 7 hours. In some embodiments, the cells are incubated with peptides for about 8 hours. In some embodiments, the cells are incubated with peptides for about 9 hours. In some embodiments, the cells are incubated with peptides for about 10 hours. In some embodiments, the cells are incubated with peptides for about 11 hours. In some embodiments, the cells are incubated with peptides for about 12 hours. In some embodiments, the cells are incubated with peptides for about 13 hours. In some embodiments, the cells are incubated with peptides for about 14 hours. In some embodiments, the cells are incubated with peptides for about 15 hours. In some embodiments, the cells are incubated with peptides for about 16 hours. In some embodiments, the cells are incubated with peptides for about 17 hours. In some embodiments, the cells are incubated with peptides for about 18 hours. In some embodiments, the cells are incubated with peptides for about 19 hours. In some embodiments, the cells are incubated with peptides for about 20 hours. In some embodiments, the cells are incubated with peptides for about 21 hours. In some embodiments, the cells are incubated with peptides for about 22 hours. In some embodiments, the cells are incubated with peptides for about 23 hours. In some embodiments, the cells are incubated with peptides for about 24 hours. In some embodiments, the cells are incubated with peptides for about 25 hours. In some embodiments, the cells are incubated with peptides for about 26 hours. In some embodiments, the cells are incubated with peptides for about 27 hours. In some embodiments, the cells are incubated with peptides for about 28 hours. In some embodiments, the cells are incubated with peptides for about 29 hours. In some embodiments, the cells are incubated with peptides for about 30 hours. In some embodiments, the method comprises an overnight incubation of the cells with the peptides at 37° C. and 5% CO₂. In some embodiments, the method comprises incubating the cells with the peptides for about 30 minutes to about 30 hours at about 37° C. and 5% CO₂ in the absence of exogenous cytokines. In some embodiments, the method does not comprise a wash step. In some embodiments, the method comprises dividing the culture into two or more individual cultures after the cells are incubated with the peptides for about 8 to about 30 hours at about 37° C. and 5% CO₂ in the absence of exogenous cytokines. In some embodiments, the cells are incubated for an additional 5 to 14 days at about 37° C. and 5% CO₂ after addition of one or more exogenous cytokines.

### Cytokines

In embodiments, the methods presented herein comprise incubation with exogenous cytokines that are suitable for stimulating and/or activating T cell expansion and/or differentiation. A surprising aspect of the present invention is that pre-incubation of the cells with the one or more peptides prior to addition of one or more exogenous cytokines significantly improved various aspects of the tumor antigen specific T cells generated by the process, including antigen specificity and phenotype. In one aspect, the present disclosure provides methods wherein the population of mononuclear cells is incubated with one or peptides from one or more tumor antigens in the absence of exogenous cytokines. In some embodiments, the population is cultured for about 30 minutes to about 30 hours in the absence of exogenous cytokines. In some embodiments, the population is cultured for about 8 hours to about 24 hours in the absence of exogenous cytokines. In some embodiments, cells are cultured in the absence of exogenous cytokines for at least about 30 minutes. In some embodiments, cells are cultured in the absence of exogenous cytokines for at least about 1 hour. In some embodiments, cells are cultured in the absence of exogenous cytokines for at least about 2 hours. In some embodiments, cells are cultured in the absence of exogenous cytokines for at least about 3 hours. In some embodiments, cells are cultured in the absence of exogenous cytokines for at least about 4 hours. In some embodiments, cells are cultured in the absence of exogenous cytokines for at least about 5 hours. In some embodiments, cells are cultured in the absence of exogenous cytokines for at least about 5 hours. In some embodiments, cells are cultured in the absence of exogenous cytokines for at least about 7 hours. In some embodiments, cells are cultured in the absence of exogenous cytokines for at least about 8 hours. In some embodiments, cells are cultured in the absence of exogenous cytokines for at least about 9 hours. In some embodiments, cells are cultured in the absence of exogenous cytokines for at least about 10 hours. In some embodiments, cells are cultured in the absence of exogenous cytokines for at least about 11 hours. In some embodiments, cells are cultured in the absence of exogenous cytokines for at least about 12 hours. In some embodiments, cells are cultured in the absence of exogenous cytokines for at least about 13 hours. In some embodiments, cells are cultured in the absence of exogenous cytokines for at least about 14 hours. In some embodiments, cells are cultured in the absence of exogenous cytokines for at least about 15 hours. In some embodiments, cells are cultured in the absence of exogenous cytokines for at least about 16 hours. In some embodiments, cells are cultured in the absence of exogenous cytokines for at least about 17 hours. In some embodiments, cells are cultured in the absence of exogenous cytokines for at least about 18 hours. In some embodiments, cells are cultured in the absence of exogenous cytokines for at least about 19 hours. In some embodiments, cells are cultured in the absence of exogenous cytokines for at least about 20 hours. In some embodiments, cells are cultured in the absence of exogenous cytokines for at least about 21 hours. In some embodiments, cells are cultured in the absence of exogenous cytokines for at least about 22 hours. In some embodiments, cells are cultured in the absence of exogenous cytokines for at least about 23 hours. In some embodiments, cells are cultured in the absence of exogenous cytokines for at least about 24 hours. In some embodiments, cells are cultured in the absence of exogenous cytokines for at least about 25 hours. In some embodiments, cells are cultured in the absence of exogenous cytokines for at least about 26 hours. In some embodiments, cells are cultured in the absence of exogenous cytokines for at least about 27 hours. In some embodiments, cells are cultured in the absence of exogenous cytokines for at least about 28 hours. In some embodiments, cells are cultured in the absence of exogenous cytokines for at least about 29 hours. In some embodiments, cells are cultured in the absence of exogenous cytokines for at least about 30 hours.

In embodiments, after the incubation of cells and peptide in the absence of exogenous cytokines, one or more exogenous cytokines are added to the culture. In embodiments, the exogenous cytokines include IL-6, IL-7, IL-15, IL-12, or any combination thereof. In embodiments, the exogenous cytokines added to the culture consist of IL-6, IL-7, IL-15, and IL-12. In embodiments, IL-6 is added to the culture at a concentration of about 10 ng/mL to about 100 ng/mL, for example, about 10 ng/mL to about 50 ng/mL, or about 25 ng/mL to about 75 ng/mL, or about 50 ng/mL to about 100 ng/mL. In embodiments, IL-7 is added to the culture at a concentration of about 1 ng/mL to about 10 ng/mL, for example, about 1 ng/mL to about 5 ng/mL, or about 2.5 ng/mL to about 7.5 ng/mL, or about 5 ng/mL to about 10 ng/mL. In embodiments, IL-15 is added to the culture at a concentration of about 1 ng/mL to about 5 ng/mL, for example, about 1 ng/mL to about 2.5 ng/mL, or about 2.5 ng/mL to about 5 ng/mL. In embodiments, IL-12 is added to the culture at a concentration of about 10 to about 100 IU/mL, for example, about 10 IU/mL to about 55 IU/mL, or about 50 IU/mL to about 100 IU/mL, or about 25 IU/mL to about 75 IU/mL. In embodiments, once the cytokines are added to the culture, the culture is not disturbed until the MultiTAA-specific T cells are harvested. In embodiments, the cells are harvested after an incubation period of about 5 to about 14 days, for example, about 5 to about 10 days, for example, about 5, about 6, about 7, about 8, about 9, about 10, about 11, about 12, about 13, or about 14 days.

### MultiTAA-Specific T Cell Populations and Compositions

In one aspect, the present disclosure provides a population of antigen-specific T cells (also called tumor antigen-specific T cells, MultiTAA-specific T cells) generated by the methods described herein. In one aspect, the present disclosure provides a population of antigen-specific T cells comprising between about 80% to about 99% CD3+ T cells, <1% CD14+ monocytes, <10% CD19+ B cells, and <10% CD3-CD56+NK cells.

T cells comprise distinct subsets, which may arise via differentiation and may differ in activity, longevity, and expansion kinetics. T cell subsets comprise CD4+ T cells and CD8+ T cells, naïve T cells, central memory (T_{CM}), and effector memory (T_{EM}) cells. T cell subsets may be detected and measured by methods known to those of ordinary skill in the art, e.g. flow cytometry methods following labeling of distinguishing phenotypic markers (e.g., CD4, CD8, CD45RA, CD45RO, etc.) with fluorescently labeled antibodies.

In embodiments, the present disclosure provides a population of antigen-specific T cells comprising at least about 20% central memory T cells (T_{CM}). In embodiments, the tumor antigen-specific T cells comprise at least about 20% naïve T cells. In embodiments, the tumor antigen-specific T cells comprise about 40% effector memory T cells (T_{EM}). In embodiments, the tumor antigen-specific T cells comprise no more than 10% terminally differentiated effector memory cells expressing CD45RA (T_{EMRA}).

In embodiments, the present disclosure provides a population of antigen-specific T cells that exhibits a higher magnitude of antigen-specific response relative to populations of antigen-specific T cells produced by prior methods. For example, the population of antigen-specific T cells exhibits a magnitude of specificity (combined reactivity against each target antigen) of at least about 500 SFU/2×10⁵ cells as measured by IFNγ ELISpot. Functionality and antigen specificity of T cells can be determined by methods known in the art including methods for measuring proliferation and/or cytokine expression, e.g. IFNγ expression. IFNγ expression can be assessed, for example, by ELISA, ELISpot, or flow cytometry analyses.

T cell populations may be characterized by the diversity of antigen specificity (e.g., the number of tumor antigens, among those represented by the one or more peptides incubated with the mononuclear cells, for which the T cell product has activity). Higher diversity of antigen specificity may be a desirable characteristic of T cell populations. In some embodiments, the tumor antigen-specific T cells produced by the methods herein exhibit antigen specificity for at least 50%, at least 60%, at least 70%, at least 75%, at least 80%, at least 85%, at least 90%, or at least 95% of the tumor antigens represented by the one or more peptides used in the initial culture. In some embodiments, the tumor antigen-specific T cells produced by the methods herein exhibit antigen specificity for at least 75% of the tumor antigens represented by the one or more peptides used in the initial culture. In some embodiments, the tumor antigen-specific T cells produced by the methods herein exhibit antigen specificity for each of the tumor antigens represented by the one or more peptides. In embodiments, increasing the number of tumor antigens incubated with the mononuclear cells surprisingly increases the antigen specificity as well as antigen diversity of the tumor antigen-specific T cells produced.

In some embodiments, the population comprises T cells that exhibit activity against one or more peptides of one or more antigens that the population has not been previously incubated with. In some embodiments, said activity against the one or more peptides of one or more antigens that the population has not been previously incubated with is a result of epitope spreading. In some embodiments, the population comprises a magnitude of specificity against the one or more peptides of one or more antigens that the population has not been previously incubated which, cumulatively, is at least about 100 SFU/2×10⁵ cells as measured by IFN-γ ELISpot. In some embodiments, the population comprises a magnitude of specificity against the one or more peptides of one or more antigens that the population has not been previously incubated which, cumulatively, is at least about 30 SFU/2×10⁵ cells as measured by IFN-γ ELISpot.

In embodiments, the present disclosure provides a composition comprising a population of MultiTAA-specific T cells obtained by the methods provided herein. In embodiments, this composition further comprises a pharmaceutically acceptable buffer or excipient. In further embodiments, the

### T-Cell Products

In one aspect, the present disclosure provides a MultiTAA-specific T cell product (also called a T cell product herein) generated by the methods described herein. In one aspect, the present disclosure provides a T cell product generated by the methods described herein, comprising between about 80% to about 99% CD3+ T cells, <1% CD14+ monocytes, <10% CD19+ B cells, and <10% CD3-CD56+NK cells.

In embodiments, the present disclosure provides a MultiTAA-specific T cell product comprising at least about 20% central memory T cells (T_{CM}). In embodiments, the tumor antigen-specific T cells comprise at least about 20% naïve T cells. In embodiments, the tumor antigen-specific T cells comprise about 40% effector memory T cells (T_{EM}). In embodiments, the tumor antigen-specific T cells comprise no more than 10% terminally differentiated effector memory cells expressing CD45RA (T_{EMRA}). T_{CM} are generally regarded to have greater potential for proliferation and persistence in vivo compared to terminally differentiated T cells; therefore, the T cell products described herein seem to present favorable characteristics for treatment.

In embodiments, the present disclosure provides a MultiTAA-specific T cell product that exhibits a higher magnitude of antigen-specific response relative to MultiTAA-specific T cell products produced by prior methods. For example, the MultiTAA-specific T cell product exhibits a magnitude of specificity (combined reactivity against each target antigen) of at least about 500 SFU/2×10⁵ cells as measured by IFNγ ELISpot. For example, in embodiments, the MultiTAA-specific T cell product exhibits a magnitude of specificity (combined reactivity against each target antigen) of at least about 700 SFU/2×10⁵ cells, at least about 1000 SFU/2×10⁵ cells, or at least about 1200 SFU/2×10⁵ cells, as measured by IFNγ ELISpot.

In some embodiments, the T cell products produced by the methods herein exhibit antigen specificity for at least 50%, at least 60%, at least 70%, at least 75%, at least 80%, at least 85%, at least 90%, or at least 95% of the tumor antigens represented by the one or more peptides used in the initial culture. In some embodiments, the T cell products produced by the methods herein exhibit antigen specificity for at least 75% of the tumor antigens represented by the one or more peptides used in the initial culture. In some embodiments, the T cell products produced by the methods herein exhibit antigen specificity for each of the tumor antigens represented by the one or more peptides. In embodiments, increasing the number of tumor antigens incubated with the mononuclear cells surprisingly increases the antigen specificity as well as antigen diversity of the tumor antigen-specific T cells produced.

In some embodiments, the T cell product comprises T cells that exhibit activity against one or more peptides of one or more antigens not used to generate the T cell product. In some embodiments, the T cell product comprises a magnitude of specificity against the one or more peptides of one or more antigens not used to generate the T cell product which, cumulatively, is at least about 100 SFU/2×10⁵ cells as measured by IFN-γ ELISpot. In some embodiments, the T cell product comprises a magnitude of specificity against the one or more peptides of one or more antigens not used to generate the T cell product which, cumulatively, is at least about 30 SFU/2×10⁵ cells as measured by IFN-γ ELISpot.

In some embodiments, said activity against the one or more peptides of one or more antigens that the population has not been previously incubated with is a result of epitope spreading.

Epitope spreading occurs when T cells targeting specific antigens destroy them, exposing new “non-targeted” epitopes/antigens, which recruits and expands T cells specific for non-target antigens to help eliminate the tumor, thus limiting tumor escape. Epitope spreading in this context was previously thought to be a result of the endogenous immune system from the patient. Surprisingly, the present inventors found that epitope spreading comes from an extension of the MultiTAA-specific T cell product itself. Without wishing to be bound by theory, cells specific for non-target antigens within the final drug product are likely what contributes to epitope spreading to eliminate tumor in vivo.

### Cryopreservation

In some embodiments, the method further comprises cryopreserving the mononuclear cells, and then thawing the cryopreserved cells prior to placing them in the vessel. In some embodiments, the method further comprises cryopreservation of the MultiTAA-specific T cell product. Cryopreservation may be performed by placing the mononuclear and/or MultiTAA-specific T cells into a cryopreservation media or freezing solution, e.g., CryoStor® CS10 or a derivative thereof, Hyperthermasol, or any freezing solution comprising about 5 to about 15% DMSO. Thus, in some embodiments, the present disclosure provides mononuclear cells and/or MultiTAA-specific T cells as described herein, in a cryopreserved state. In order to utilize mononuclear and/or MultiTAA-specific T cells after cryopreservation, for example for use of mononuclear cells to incubate with peptides or for use of MultiTAA-specific T cells in treating a patient, the cells are removed from the freezer and thawed in an about 37° C. water bath until a majority of the solution is thawed. The cells may then be resuspended in media, optionally washed before use. In some embodiments, the thawed mononuclear cells are rested for about 4 to about 48 hours prior to placing them in the vessel. In some embodiments, the mononuclear cells and/or thawed MultiTAA-specific T cells are prepared for use by counting and determining cell viability.

In embodiments, the present disclosure provides off the shelf tumor antigen-specific T cell products. Such products remove the need for donor identification, cell procurement, manufacture of the T cell product, and analysis of the T cell product steps that have to occur between patient identification/enrollment and patient treatment, and thus provide a significantly shortened time to patient treatment. Instead of 14, 21, 28, 35, or longer (for example) time to treatment, patients can be treated with an off the shelf tumor antigen-specific T cell product within about 72 hours of identification of the patient. In embodiments, the methods for generating a MultiTAA-specific T cell product provided herein further comprise a step of HLA typing a donor of the mononuclear cells used to generate the MultiTAA-specific T cell product.

### Methods of Treatment

In one aspect, the present disclosure provides methods for treating a patient having cancer. In some embodiments, the methods provided herein include administering to a patient in need thereof a pharmaceutical composition comprising the MultiTAA-specific T cells provided herein. In an embodiment, the pharmaceutical composition is a suspension of MultiTAA-specific T cells in a pharmaceutically acceptable buffer or excipient. In some embodiments, the methods comprise administering to a patient a tumor antigen-specific T cell product provided herein, wherein the product is administered to the patient at a dose of about 50×10⁶ cells to about 300×10⁶ cells per treatment. For example, in embodiments, the product is administered to the patient at a dose of about 100×10⁶ to about 200×10⁶ cells per dose. In embodiments, the product is administered to the patient at a dose of about 100×10⁶ or about 200×10⁶ cells per dose. In embodiments, the product is administered to the patient by any suitable route, especially intravenous infusion or intra-arterial infusion. In embodiments, the infusion may last approximately 15, 30, 45, 60, or 90 minutes per dose. In embodiments, the MultiTAA-specific T cell product or pharmaceutical composition is administered in a single dose or in multiple doses.

In embodiments, the cancer is a lymphoma or leukemia. In embodiments, the cancer is a solid tumor. In embodiments, the patient has a cancer selected from acute myeloid leukemia (AML), myelodysplastic syndrome (MDS), acute lymphoblastic leukemia (ALL), multiple myeloma, non-Hodgkin's lymphoma, sarcoma, breast cancer, and pancreatic cancer.

Thus, in embodiments, the present disclosure provides methods for treating a patient having a cancer with an off the shelf MultiTAA-specific T cell product provided herein, wherein the patient is HLA typed or wherein the HLA type of the patient is known, and a MultiTAA-specific T cell product is selected from a bank of previously generated MultiTAA-specific T cell products to achieve a desired level of HLA matching between donor and recipient. In embodiments, the donor and recipient have at least one HLA match, at least two HLA matches, or at least 3 HLA matches. In embodiments, the method comprises administering to a patient an off the shelf MultiTAA-specific T cell product provided herein and further comprises treating the patient with one or more additional off the shelf MultiTAA-specific T cell products. In embodiments, the one or more additional T cell products (e.g., second T cell product) may be administered to the patient at least about 2, about 3, about 4, about 5, or about 6 weeks after treatment with the previous T cell product was administered. In embodiments, the one or more additional treatments may be with the same off the shelf MultiTAA-specific T cell product as the previous one or more treatments (e.g., a MultiTAA-specific T cell product derived from cells from the same donor), or may be with a different off the shelf MultiTAA-specific T cell product relative to the one or more previous treatments (e.g., the one or more additional treatments may be treatments with one or more MultiTAA-specific T cell products generated from the cells of one or more different donors). In embodiments, the selection of a different MultiTAA-specific T cell product for a second or further treatment may be suitable for use in a patient that mounted an immune response against the first MultiTAA-specific T cell product and/or has rejected the first MultiTAA-specific T cell product or is suspected of having rejected the first MultiTAA-specific T cell product.

In embodiments, the second population of antigen-specific T cells (second treatment) in an autologous setting is generated as follows: (i) a first population of mononuclear cells is obtained from a patient; (ii) a first population of antigen-specific T cells is generated according to the methods described herein; (iii) the first population of antigen-specific T cells is administered to the patient; (iv) at least about 4, about 5, or about 6 weeks after administration of the first population of antigen-specific T cells, a second population of mononuclear cells is obtained from the patient; (v) a second population of antigen-specific T cells is generated according to the methods described herein, and (vi) the second population of antigen-specific T cells is administered to the patient. This second antigen-specific T cell product may contain an amplified number of the antigen specific cells compared to the first antigen-specific T cell product, providing a more potent second dose.

In an aspect, the present disclosure provides MultiTAA-specific T cells that are allogeneic to the recipient patient and that effectively treat a cancer without significant effect of a graft-versus-host disease (GVHD) response. In general, a GVHD response would be expected to occur in a patient population receiving allogeneic tumor antigen-specific T cells for treatment of a cancer because the patient is not immuno-compromised. Without wishing to be bound by theory, the surprising lack of GVHD response in recipients of the MultiTAA-specific T cells provided herein may be due to the enrichment of tumor antigen-specific cells that is achieved by the compositions and methods provided herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, or up to 1% of a given value. Alternatively, particularly with respect to biological systems or processes, the term can mean within an order of magnitude, e.g., within 5-fold or within 2-fold, of a value. Where particular values are described in the application and claims, unless otherwise stated the term “about” meaning within an acceptable error range for the particular value should be assumed.

As used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

In the present description, any concentration range, percentage range, ratio range, or integer range is to be understood to include the value of any integer within the recited range and, when appropriate, fractions thereof (such as one tenth and one hundredth of an integer), unless otherwise indicated. The terms “about” and “approximately” mean within a statistically meaningful range of a value. Such a range can be within an order of magnitude, preferably within 20%, more preferably still within 10%, and even more preferably within 5% of a given value or range. In certain embodiments, the term “about”, when immediately preceding a number or numeral, means that the number or numeral ranges plus or minus 10%. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Unless otherwise indicated, all terms used herein have the same meaning as they would to one skilled in the art and the practice of the present invention will employ, conventional techniques of microbiology and recombinant DNA technology, which are within the knowledge of those of skill of the art.

The term “mononuclear cells” refers to cells having a round nucleus, such as those isolated from peripheral blood of a patient or donor. Mononuclear cells may be a population of white blood cells or peripheral blood mononuclear cells (PBMCs), and can be obtained using known methods such as Ficoll-Hypaque density gradient method. Mononuclear cells include monocytes, lymphocytes, T cells, B cells, and NK cells.

The terms “subject,” “individual,” and “patient” are used interchangeably herein. In embodiments, the subject is a mammal, for example, a mouse, a rat, a rabbit, a cat, a dog, a pig, a sheep, a horse, a non-human primate (e.g., cynomolgus monkey, chimpanzee), or a human. The term “donor” refers to a subject, individual, or patient from whom mononuclear cells are obtained.

As used herein, the term “exogenous” refers to a material introduced into a cell, cell culture, vessel, device, suspension, composition, and the like, which was produced outside of the cell, cell culture, vessel, device, suspension, or composition. The term “endogenous” refers to any material that was produced from inside a cell, cell culture, vessel, device, suspension, or composition.

“T cell” is a term commonly employed in the art and intended to include thymocytes, immature T lymphocytes, mature T lymphocytes, resting T lymphocytes or activated T lymphocytes. A T cell can be a T helper (Th) cell, for example a T helper 1 (Th1) or a T helper 2 (Th2) cell. The T cell can be a CD4+ T cell, CD8+ T cell, CD4+CD8+ T cell, CD4-CD8− T cell or any other subset of T cells.

The term “antigen specific T cell” as employed herein is intended to refer to T cells that recognize a particular antigen and respond, for example by proliferating and/or producing cytokines in response thereto. The terms “tumor antigen-specific T cells” or “tumor antigen-specific T cell product” or “tumor antigen-specific T cell population” and the like are used interchangeably herein with the terms “MultiTAA-specific T cells” or “MultiTAA-specific T cell product” and the like. The terms “tumor specific antigen” and “tumor antigen” are used interchangeably herein with the term “tumor associated antigen” or “TAA”.

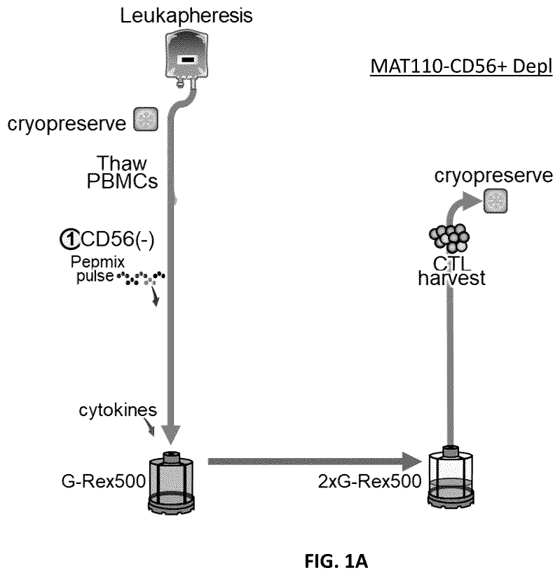

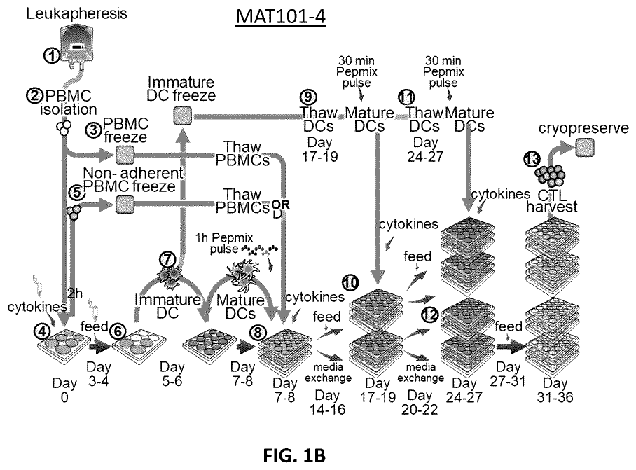

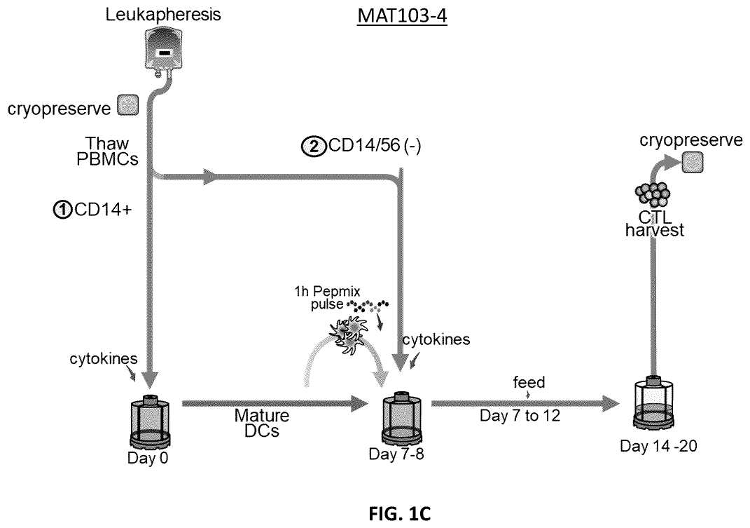

The term “MAT101-4” is used herein to refer to a MultiTAA-specific T cell manufacturing process as shown schematically in FIG. 1B. The term “MAT103-4” is used herein to refer to a MultiTAA-specific T cell manufacturing process as shown schematically in FIG. 1C. The term “MAT110-CD56+Depl” is used herein to refer to a MultiTAA-specific T cell manufacturing process as shown schematically in FIG. 1A. The MAT110-CD56+Depl process involves direct stimulation of PBMCs with peptide, in a vessel having at least one gas permeable surface. The term “MAT210-CD56+Depl-CD19+Depl” is used herein to refer to a MultiTAA-specific T cell manufacturing process identical to MAT110-CD56+Depl except that the mononuclear cells are depleted of CD19+ cells as well as CD56+ cells prior to incubation with peptides. Unlike the MAT101-4 and MAT110-No Depl processes, the MAT110-CD56+Depl and MAT210-CD56+Depl-CD19+Depl processes comprise a step of incubating the peptide mix with PBMCs in the absence of exogenous cytokines prior to addition of one or more exogenous cytokines. The MAT110-CD56+Depl and MAT210-CD56+Depl-CD19+Depl processes also span fewer total days compared to the MAT101-4 or MAT103-4 process, i.e., about 5 to about 14 days.

In embodiments, the identity and/or phenotype of cells is measured to determine characteristics of the multiTAA-specific T cells provided herein, or to determine characteristics of the cells used as a starting material to generate the multiTAA-specific T cells provided herein. Expression markers such as, but not limited to, CD3, CD4, CD8, CD56, CD19, CD14, and any other marker disclosed herein or known in the art, may be measured by flow cytometry methods. For example, antibodies, e.g. fluorescently labeled antibodies, may be used to label the cells which can then be analyzed using a flow cytometer. Moreover, cell number and/or cell viability can be measured using a hemocytometer, trypan blue staining, or any other method known in the art. In some embodiments, other cell markers such as regulatory markers are measured, for example to assess the functionality or exhaustion of the MultiTAA-specific T cells. Such markers maybe selected from, for example, TCRαβ, CD45RA, CD45RO, CD25, CD28, CD127, CD95, CCR7, CD62L, Lag3, TIM-3, PD-1, TIGIT, and the like.

The term “central memory T cell” is used interchangeably herein with “T_{CM}” and refers to a subset of T cells that secrete IL-2 after activation. T_{CM} cells are CD45RO+CD45RA^{lo}, CD127+(IL-7R+) and constitutively express CCR7 (CCR7^{hl}) and CD62L (CD62^{hl}). The term “effector memory T cell” is used interchangeably herein with “T^{EM}” and refers to a subset of T cells that, like central memory T cells, are CD45RO+CD45RA^{lo}, and CD127+(IL-7R+), but are CCR7^{lo}) and CD62L^{lo} Effector memory T cells re-expressing CD45RA (T_{EMRA} are CD45RO^{lo}, CD45RA^{hl}, CD62^{lo}, and CCR7^{lo}). T_{EM} and T_{EMRA} are more differentiated in terms of effector function than T_{CM}, which generally have a higher proliferative potential. The term “epitope spreading” as used herein refers to spreading or diversification of the epitope specificity of a T cell immune response. For example, in embodiments, epitope spreading refers to spreading or diversification from an initial set of specificities (e.g., specificity for the tumor antigens used to generate or expand the antigen-specific T cell product) to additional specificities, for example, other tumor antigens that were not present during the generation or initial expansion of the antigen-specific T cell product.

As used herein, the term “harvest” means to collect, to isolate, and/or to remove from the culture vessel or device. Antigen-specific T cells may be harvested using methods known to those of ordinary skill in the art, including but not limited to removal from the culture vessel or device, optionally centrifuging or using an automated cell processing device to wash cells, and optionally resuspending in fresh media, a solution suitable for counting, a solution suitable for analysis, a solution suitable for cryopreservation, or a solution suitable for administration to a patient.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment, such as cancer. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried. In embodiments, an effective amount of the multiTAA-specific T cells may be administered in either single or multiple doses by any of the accepted modes of administration of such a cell product, including intravenous injection, intra-arterial injection, intraperitoneally, parenterally, intramuscularly, intrathecally, intralymphatically, or subcutaneously.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

It is to be understood that the description above as well as the examples that follow are intended to illustrate, and not limit, the scope of the invention. Other aspects, advantages and modifications within the scope of the invention will be apparent to those skilled in the art to which the invention pertains.

### EXAMPLES

### Example 1. Development of an Improved Manufacturing Process

Known manufacturing processes for generating tumor antigen-specific T cells are costly, time-consuming, and difficult to scale to large manufacturing. The improved manufacturing process (MAT110-CD56+Depl, shown in FIG. 1A) was developed to address these limitations of prior processes (MAT101-4, shown in FIG. 1B; MAT110-No Depl or MAT103-4, shown in FIG. 1C).

### Example 2. Comparison of Starting Materials and Expanded Products

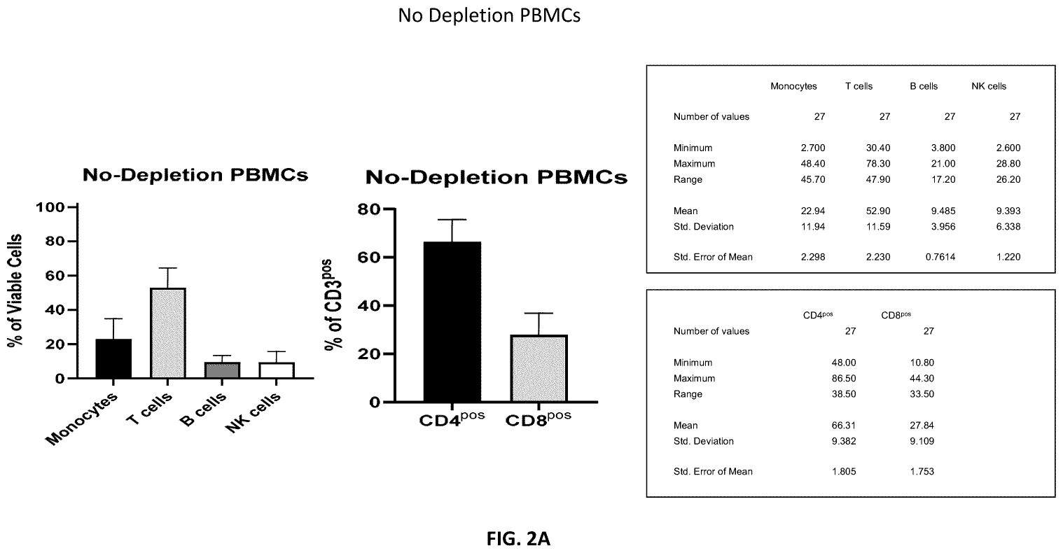

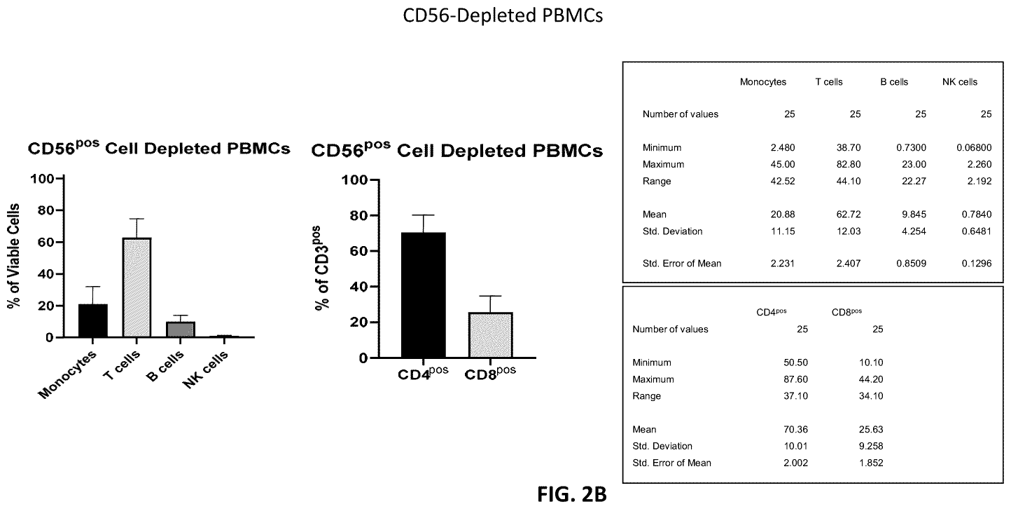

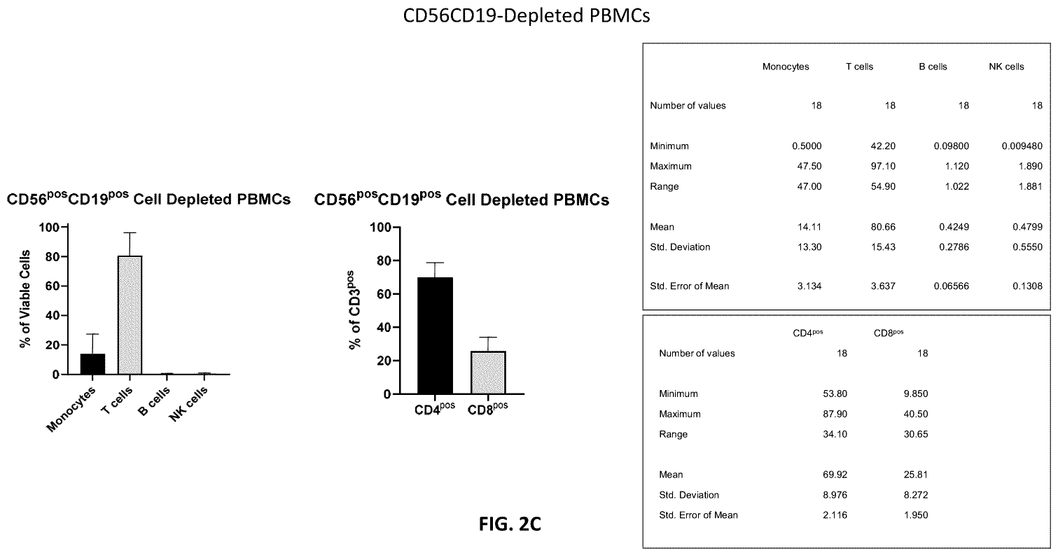

To understand if the starting cell population for generating tumor antigen-specific T cells impacted the final cell phenotype, viability, or antigen specificity, three starting populations of PBMCs were compared. PBMCs were obtained from a donor and either not subjected to depletion, depleted of CD56+ cells using the MAT110-CD56+Depl as shown in FIG. 1A (to remove NK cells), or depleted of both CD56+ cells and CD19+ cells using the MAT210-CD56+Depl-CD19+Depl method (to remove NK cells and B cells). MAT210-CD56+Depl-CD19+Depl is identical to the “MAT110-CD56+Depl” procedure except that the mononuclear cells are depleted of CD19+ cells as well as CD56+ cells prior to the peptide pulse. After the cell populations were generated, they were analyzed by flow cytometry to determine the percent (%) of viable cells that are monocytes, T cells, B cells, or NK cells (FIGS. 2A-2C). The proportion of CD4+ and CD8+ T cells in the CD3+ T cell compartment was also determined (FIGS. 2A-2C).

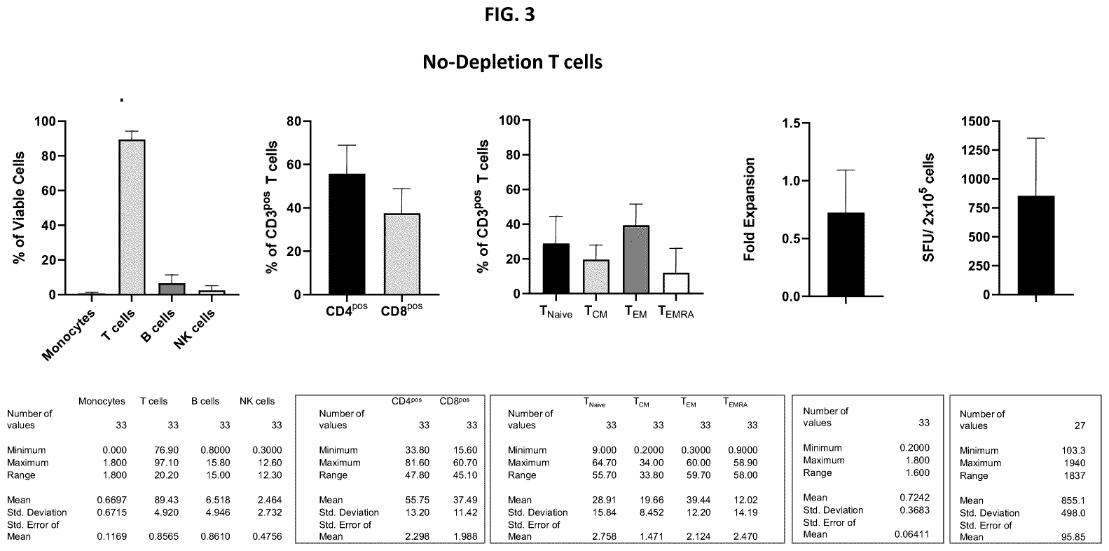

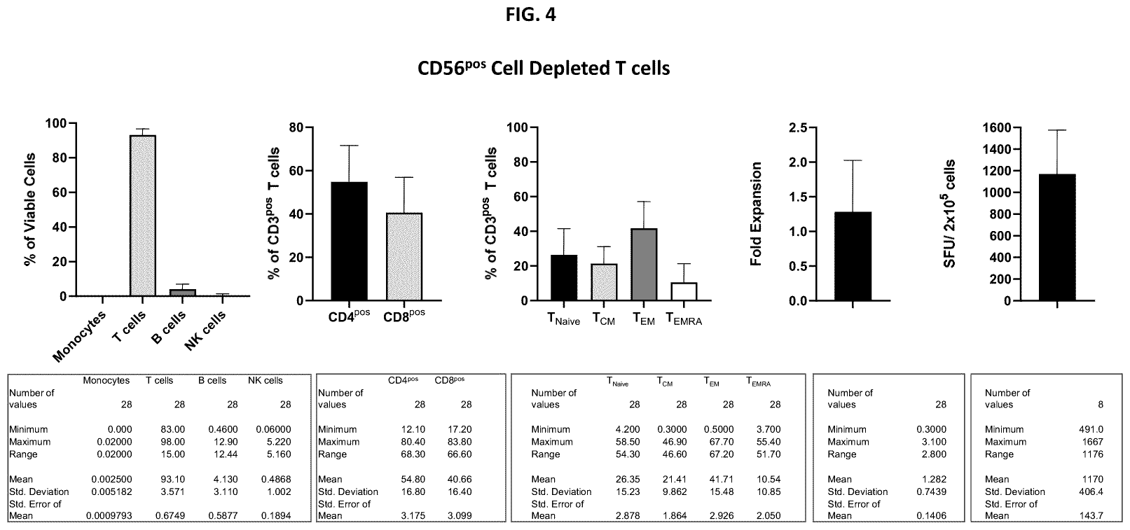

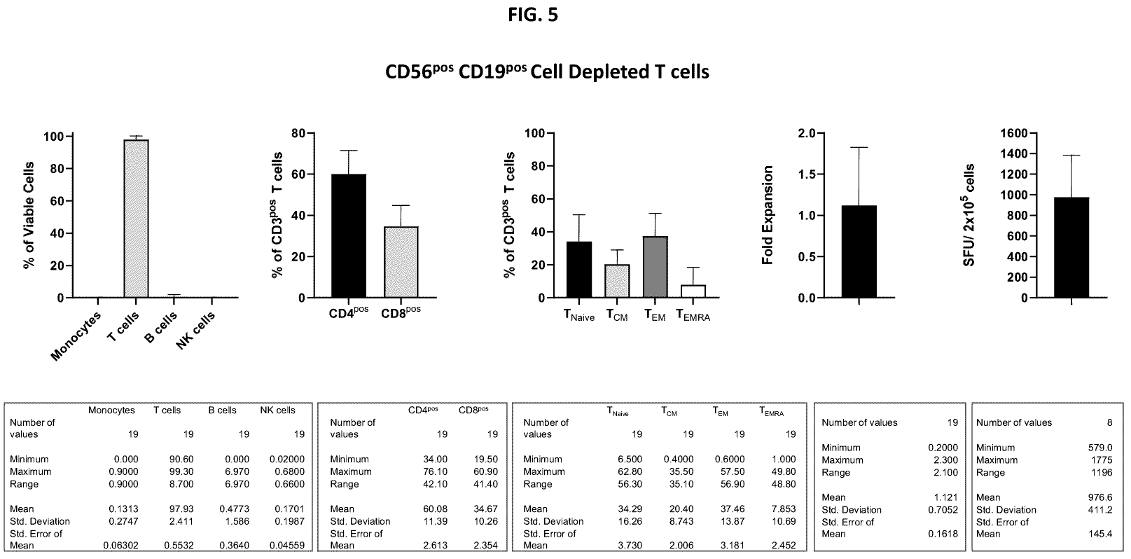

FIGS. 3, 4, and 5 provide the analysis of the tumor antigen-specific T cell products generated using the methods provided herein with non-depleted cells (FIG. 3), CD56+ depleted cells (FIG. 4), or CD56+ and CD19+ depleted cells (FIG. 5) as the starting material in the culture. The monocyte, T cell, B cell, and NK cell compartments are shown in the first bar graph in each of the figures; the percent of CD3+ T cells that are CD4+ or CD8+ are shown in the second bar graph in each of the figures; the phenotype of CD3+ T cells (Tₙₐᵢᵥₑ, T_{CM}, T_{EM}, or T_{EMRA}) are shown in the third bar graph in each of the figures; the fold expansion achieved in the cultures is shown in the fourth bar graph in each of the figures; and the total magnitude of response to the tumor antigens as measured by IFNγ ELISpot is shown in the fifth bar graph in each of the figures. The study showed that CD56+ or CD56+CD19+ depletion results in improved expansion and antigen specificity of T cells compared to no depletion.

### Example 3. Comparison of Tumor Antigen-Specific T Cells Generated Using Improved Manufacturing Process Versus Prior Processes

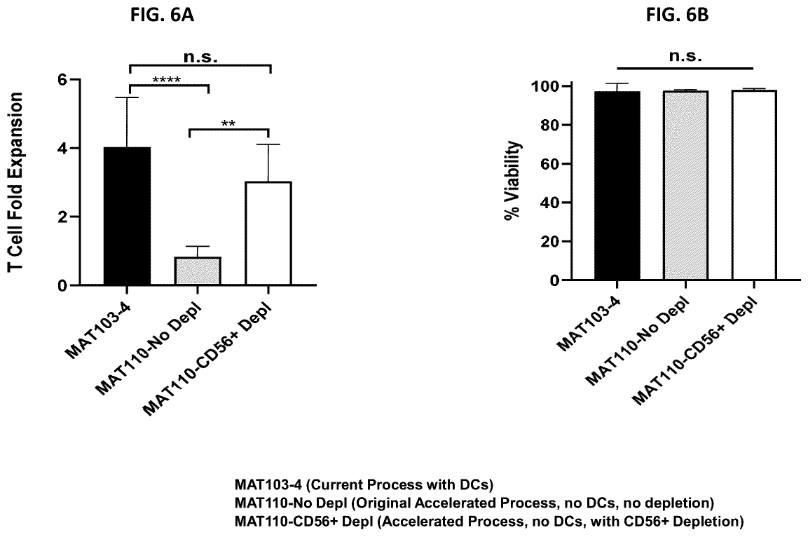

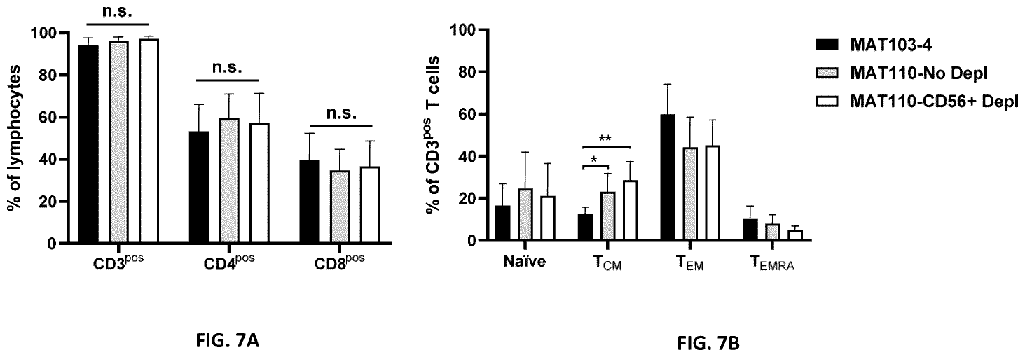

To assess the quality of the T-cell product generated using MAT110-CD56+Depl compared to those generated with prior processes, the expansion and viability of tumor antigen-specific T cells were examined. FIG. 6A shows the fold expansion of T cells for three of these methods: the MAT110-CD56+Depl method, which is an accelerated process compared to MAT103-4, showed a similar T cell fold expansion level as cells prepared by the MAT103-4 method. However, the MAT110-No Depl method had significantly lower expansion than the MAT103-4 and MAT110-CD56+Depl methods. Viability of cells was also similar across the three different methods (FIG. 6B). Next, the phenotype of the cells generated from each method was compared. FIG. 7A shows that the percent (%) of CD3+ cells and the ratio of CD4+ to CD8+ T cells was similar in the end products prepared by the three different methods. The T cell product obtained using MAT110-No Depl or MAT110-CD56+Depl had a significantly higher percentage (%) of central memory (T_{CM}) cells (FIG. 7B). For this reason, T cell products generated using MAT110-No Depl or MAT110-CD56+Depl may persist longer in vivo compared to T cell products generated using MAT103-4. Together, these data suggest that the MAT110-CD56+Depl method provides an accelerated process with favorable T cell fold expansion, viability, and potential for in vivo longevity of the T cell product.

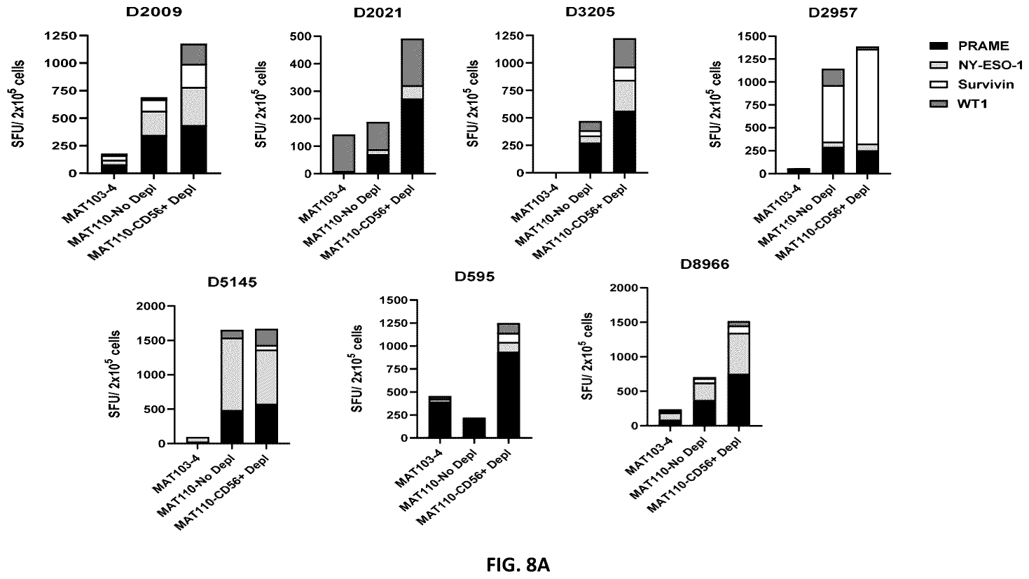

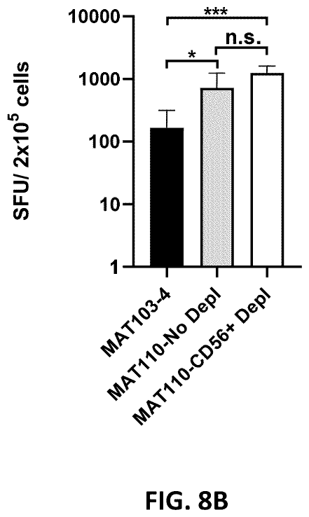

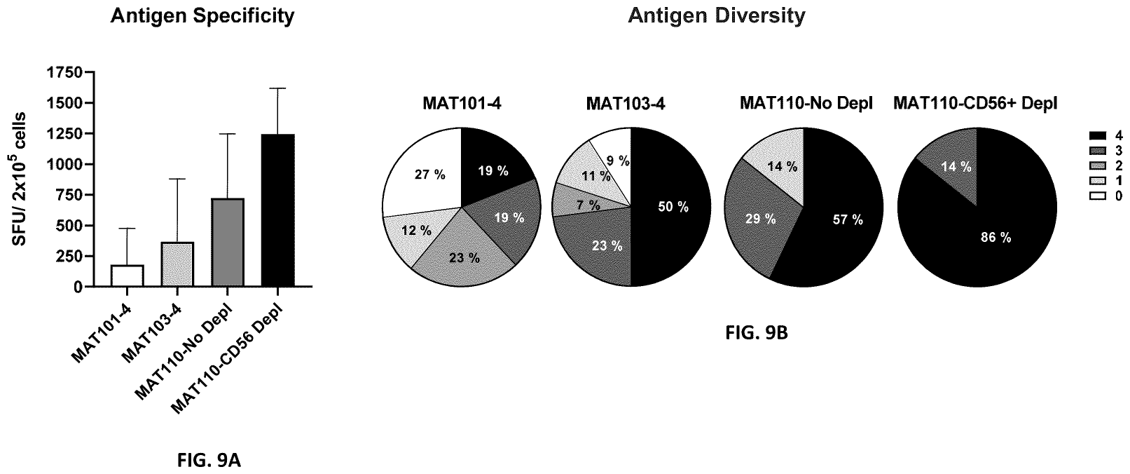

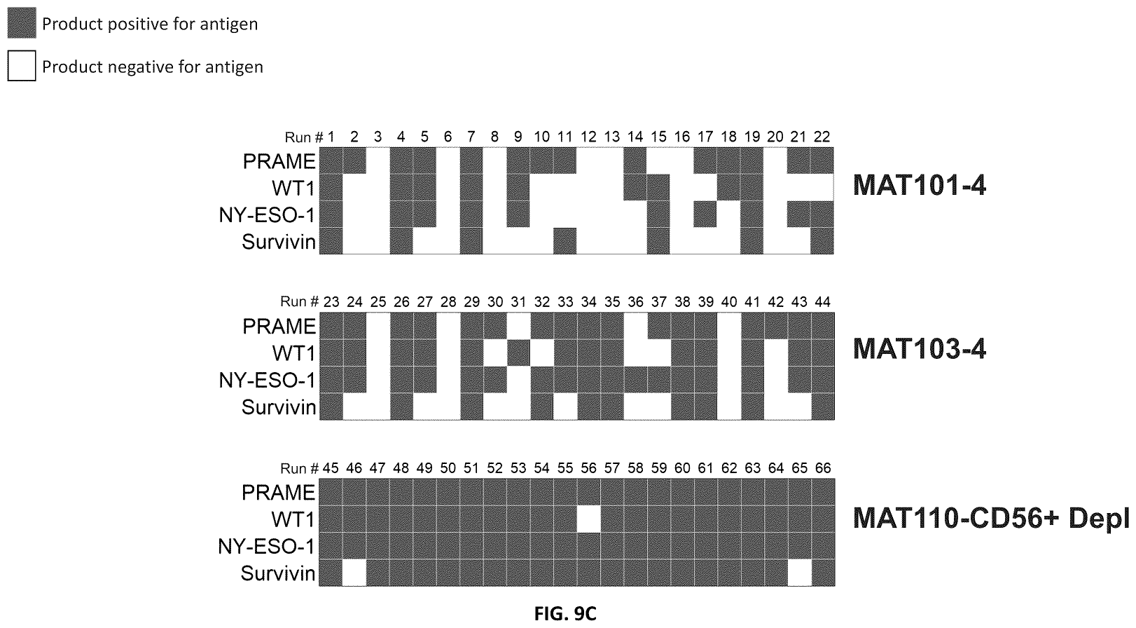

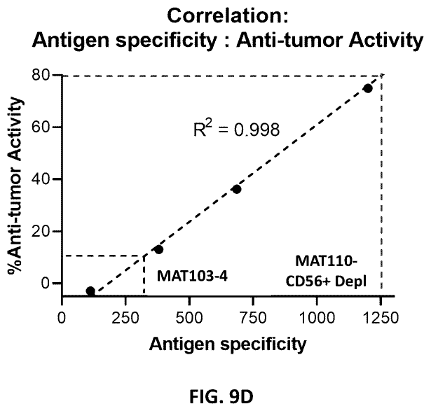

The T cells obtained by the three different methods were also assessed for antigen specificity by IFNγ ELISpot. PBMC's were collected from individual donors (i.e., donors D2009, D2021, D3205, D2957, D5145, D595, and D8966) and antigen-specific T cells were generated using the three methods described above. As shown in FIG. 8A, cells generated using the MAT110-CD56+Depl method exhibited higher antigen specificity compared to cells from the same donor produced by the MAT103-4 or MAT110-No Depl methods (i.e., higher antigen specificity compared to MAT103-4 and MAT110-No Depl). The total magnitude of antigen specificity (combined reactivity of a T cell product against each target antigen, when donor data was pooled) is shown in FIG. 8B; these data show a statistically significant increase in antigen specificity of T cell products generated using the MAT110-CD56+Depl method. The total magnitude of response is also provided in FIG. 9A. As shown in FIG. 9B, the MAT110-CD56+Depl method resulted in a higher number of T cell products with a higher number of antigen specificities, that is, a greater degree of antigen diversity. FIG. 9C further depicts the increased number of products with specificity for multiple tumor antigens that is achieved with the MAT110-CD56+Depl cells. FIG. 9D demonstrates that there is a linear correlation between anti-tumor activity (measured as change in luminescence over 6 days in firefly luciferase-expressing THP-1 cells co-cultured with MultiTAA-specific T cells) and antigen specificity (as measured by IFNγ ELISpot, SFU/2×10⁷ cells); accordingly, the increased antigen specificity achieved with the improved process provided herein are at least about four times more potent compared to prior processes.

Accordingly, the MAT110-CD56+Depl manufacturing process significantly increased the proportion of multi-targeted T cells. For example, the cells produced by the MAT110-CD56+Depl method exhibited significantly greater antigen specificity as well as diversity compared to cells produced by prior methods.

Taken together, the results of the study showed that the improved manufacturing process MAT110-CD56+Depl results in improved antigen specificity and diversity, significant fold expansion, a high level of cell viability, and a desired T cell phenotype for anti-tumor activity.

### Example 4. Analysis of Peptide Pulsing Step

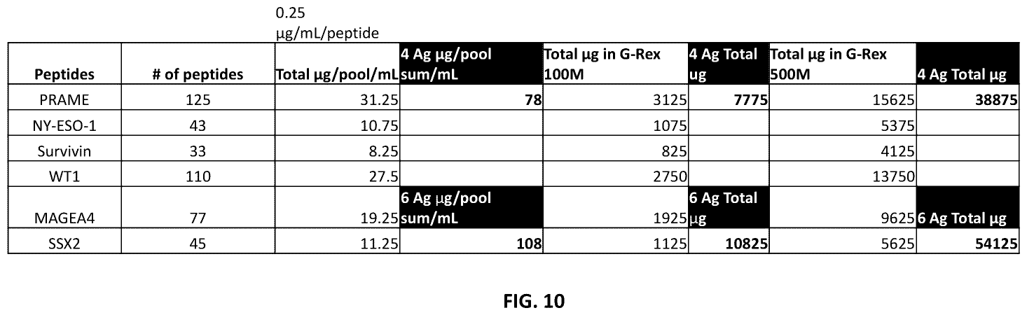

A study was conducted to assess the pre-incubation of peptide at a high concentration with cells prior to the addition of exogenous cytokines (using the MAT110-NoDepl method). PBMCs from healthy donors were pulsed with peptides from six different antigens (WT1, Survivin, NY-ESO-1, PRAME, SSX2 and MAGEA4) overnight, either in the presence or absence of cytokines (IL-6, IL-12, IL-7 and IL-15) for the manufacture of MultiTAA-specific T cells. On Day 1, cytokines were added to the “absence of cytokines” culture according to the standard MAT110-NoDepl method, while cytokines were maintained in the “presence of cytokines” culture. FIG. 10 provides an exemplary set of calculations for determining peptide amounts and concentrations for the peptide pulsing step.

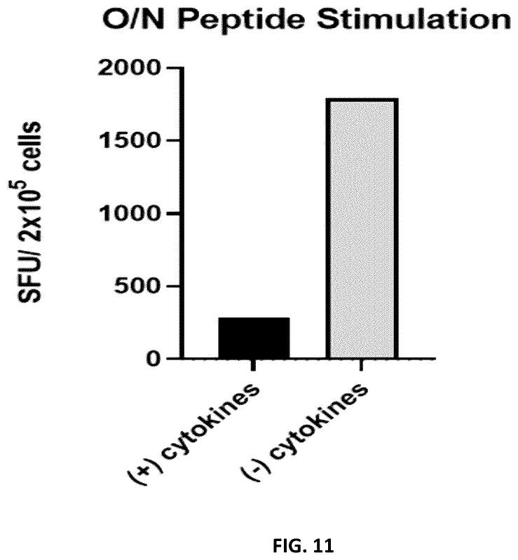

The multiple tumor antigen associated (MultiTAA)-specific T cells were then subjected to IFN-γ ELISpot analysis as a readout of antigen specificity. As shown in FIG. 11, the presence of cytokines during the peptide pulsing step significantly decreased antigen specificity, indicating that the generation of MultiTAA-specific T cells is significantly improved when the cells are incubated with peptide in the absence of cytokines for a period of time prior to the addition of the exogenous cytokines.

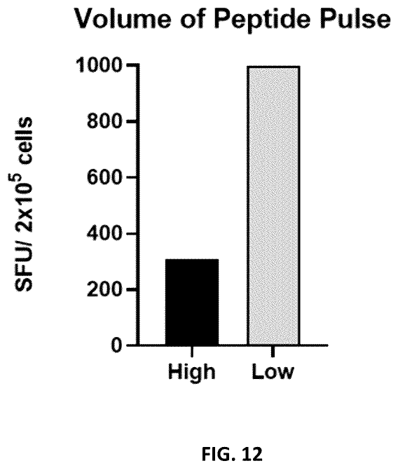

A further study was conducted to assess the effect of volume of the peptide pulsing step on antigen specificity (using the MAT110-NoDepl method). In this experiment, PBMCs from healthy donors were pulsed with peptides from 4 different antigens (WT1, Survivin, NY-ESO-1, and PRAME) overnight, either in a low volume (10 mL) or high volume (100 mL) for the manufacture of MultiTAA-specific T cells (n=3). MultiTAA-specific T cells were subjected to IFN-γ ELISpot analysis as a readout of antigen specificity. As shown in FIG. 12, antigen specificity was significantly increased in T cells produced using the low volume conditions. Without wishing to be bound by theory, the increase in antigen specificity may be due to an increase in peptide-cell contact that is achieved in a low volume peptide pulse incubation step.

Taken together, the results of the studies indicated that a peptide pulsing step carried out in the absence of exogenous cytokines and/or in a low volume (e.g., a low volume allowing contact of cells seeded at a density of about 0.1×10⁶ cells/cm² to about 1×10⁷ cells/cm² a high concentration of peptide) results in a significant improvement in antigen specificity of the resulting MultiTAA-specific T cells.

### Example 5. Comparison of Positive T Cell Selection Versus Depletion of NK Cells

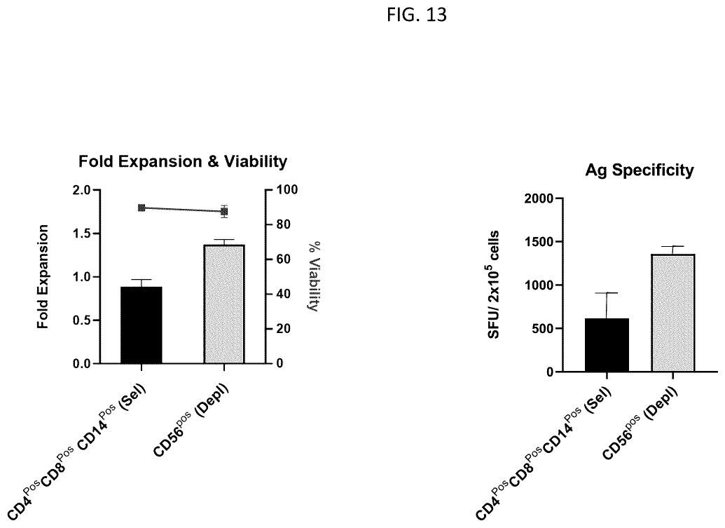

A study was conducted to compare fold expansion, viability, and antigen specificity of MultiTAA-specific T cells using starting material cells that have been positively selected for T cell markers versus cells that have been depleted of cells positive for CD56 (NK cell marker). Positively selected T cells and CD56+ depleted T cell were each incubated with tumor antigen peptides (PRAME, NY-ESO-1, Survivin, WT1) and harvested on day 9, when expansion and viability were measured. As shown in FIG. 13, positively selected T cells (CD4+CD8+CD14+) exhibited a reduction in fold expansion and antigen specificity compared to CD56+ depleted cells (MAT110-CD56+Depl method). Without wishing to be bound by theory, the positive selection of T cells via binding to CD4 and CD8 inhibits their ability to become stimulated by antigen presenting cells in the subsequent steps of the method.

### Example 6. Characterization of Antigen-Specific T Cells and Disease Progression in a Patient Recipient of MultiTAA-Specific T Cells

A study was conducted to provide various analysis of the effects of administration of a MultiTAA-specific T cell product produced by the MAT103-4 method to a cancer patient. In the study, the patient had been diagnosed with acute myeloid leukemia (AML) and had failed 5 prior lines of therapy (I+C [induction chemotherapy], FLAG-IDA [fludarabine, cytarabine (Ara-C), granulocyte-colony stimulating factor, idarubicin]+IC chemo, MEC [mitoxantrone, etoposide, and cytarabine]+IC chemo, azacytidine, and decitabine) and had a c-KIT mutation and the genetic abnormalities: t(8;21)(q22;q22.1) [RUNX1-RUNX1T1]. The matched donor's PBMCs were collected by leukapheresis, and the MultiTAA-specific T cell product was produced using peptide libraries corresponding to WT1, NY-ESO-1, Survivin, and PRAME, using the MAT103-4 method provided herein. The MultiTAA-specific T cell product (3 doses at 5×10⁶ cells per dose) was then administered to the patient, and assessments of T cell specificity, composition, phenotype, and functionality were assessed in peripheral blood samples obtained from the patient pre-infusion and at weeks 8, 12, 18, 24, and 32 post infusion. Tumor antigen expression in the bone marrow of the patient at the same time periods was also assessed by next generation sequencing (NGS). Minimal residual disease levels (% MRD) were compared to the various readouts of T cell activity and tumor antigen expression. T cell specificity for target antigens was determined by IFNγ ELISpot (SFU/2×10⁵ cells).

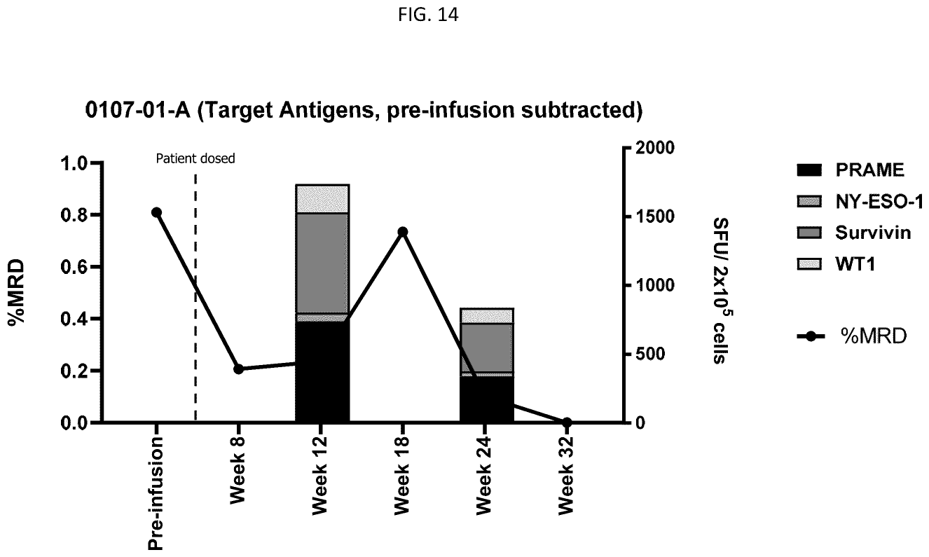

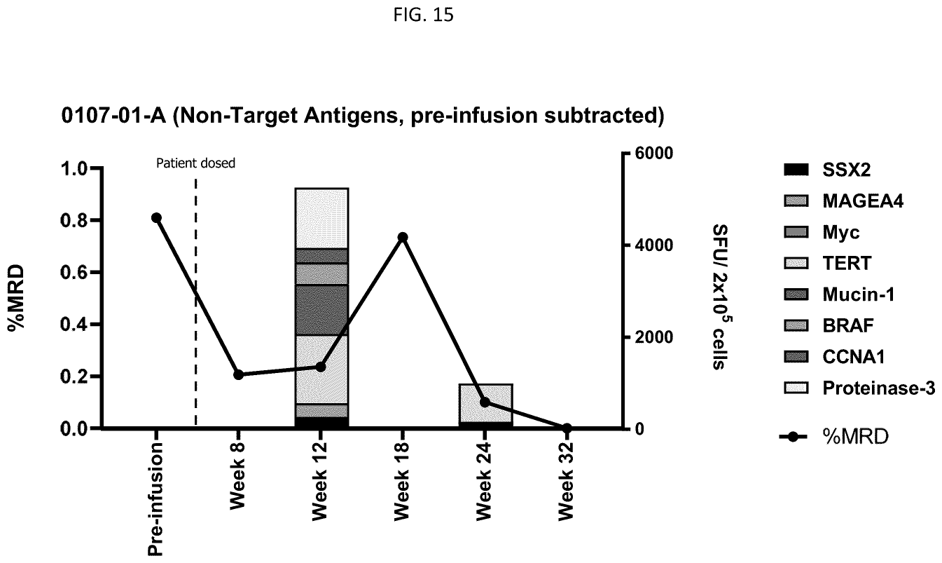

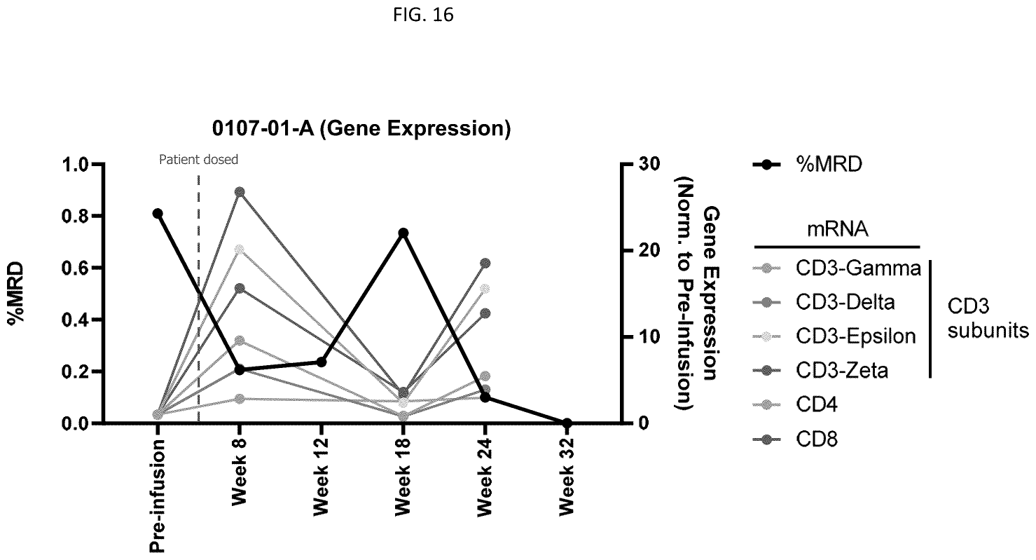

The results of the study are provided in FIGS. 14-23. FIG. 14 shows that target antigen-specific T cells (i.e., T cells specific for the target antigens used to generate the MultiTAA-specific T cell product, WT1, Survivin, NY-ESO-1, and PRAME) are present in the patient peripheral blood and inversely correlate with MRD, suggestive of anti-tumor activity. Moreover, T cells specific for additional tumor antigens not present in the set of target antigens used to generate the MultiTAA-specific T cell product were present in the patient peripheral blood, and also inversely correlate with MRD (FIG. 15). These data show epitope spreading taking place following infusion of the MultiTAA-specific T cell product, and further suggest a significant contribution of “non-target” immune responses to the anti-tumor response after infusion of the product. FIG. 16 shows T cell expression within the patient's bone marrow as detected by next generation sequencing (NGS). T cell infiltration of the bone marrow (indicating lymphocyte infiltration) inversely correlated with disease (MRD), and increased tumor burden was followed by T cell infiltration.

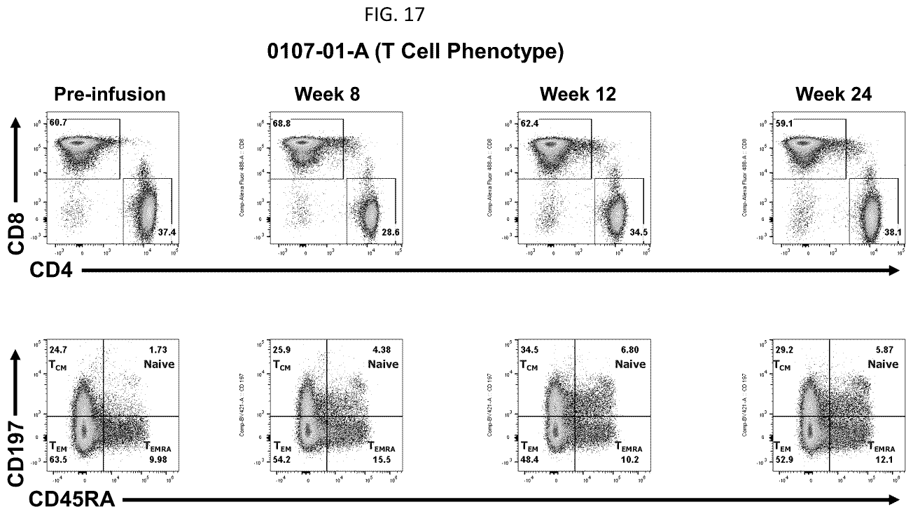

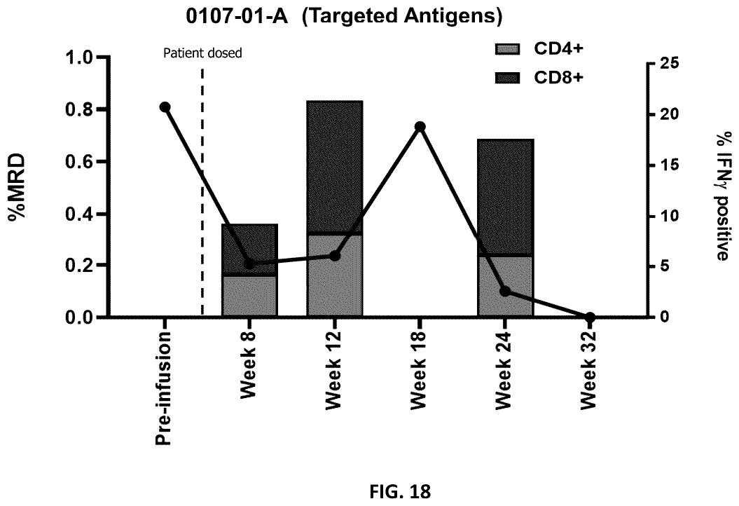

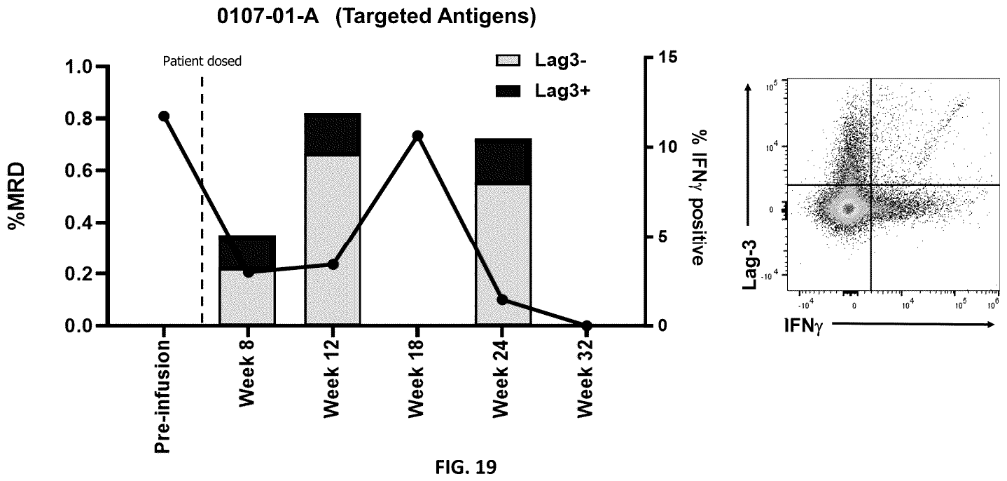

FIG. 17 shows that the T cell composition and phenotype in the patient PBMCs was relatively stable over time in the study. Functional studies showed that the target antigen-specific IFNγ production was persistent in both CD4+ and CD8+ T cells (FIG. 18). Further, most antigen-specific T cells (i.e., IFNγ+) were Lag-3 negative, thus antigen-specific T cells in the patient were protected from being exhausted (FIG. 19).

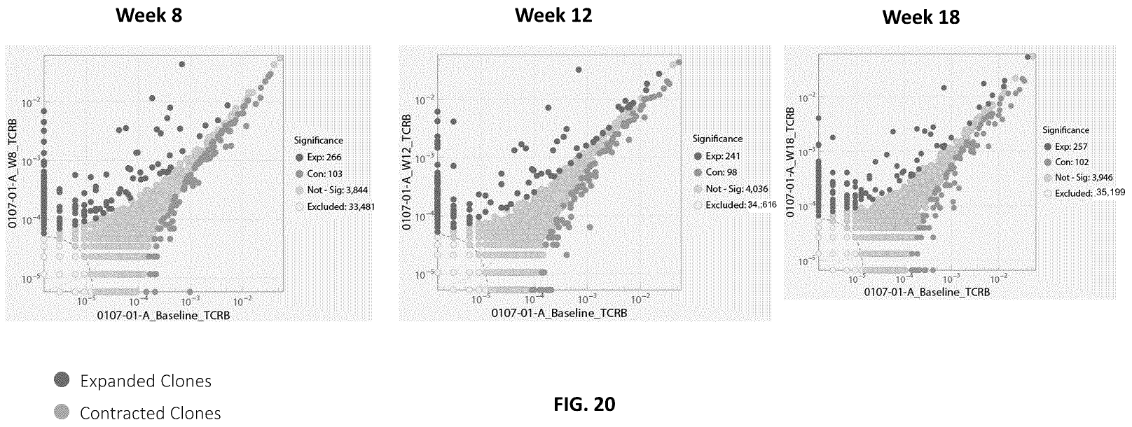

FIG. 20 shows that a significant number of different T cell clones expanded in the patient after product infusion. Clonal expansion/contraction of T cells was observed using TCR monitoring.

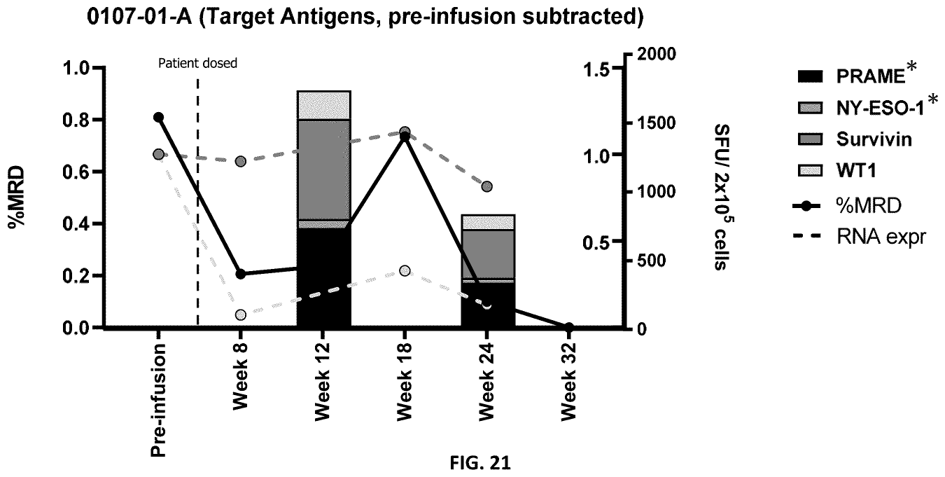

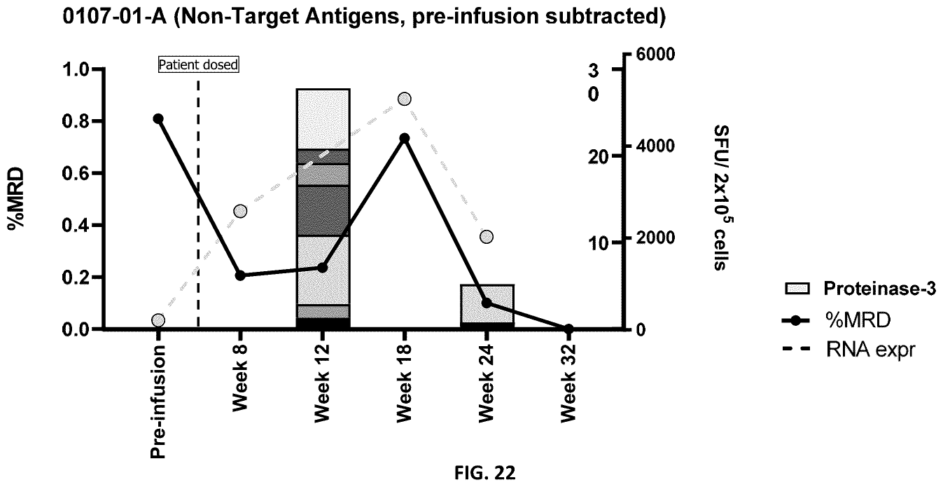

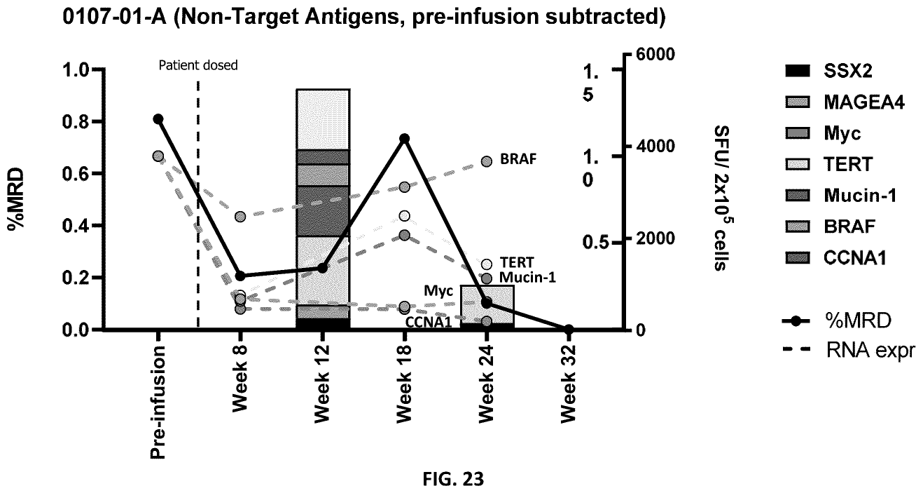

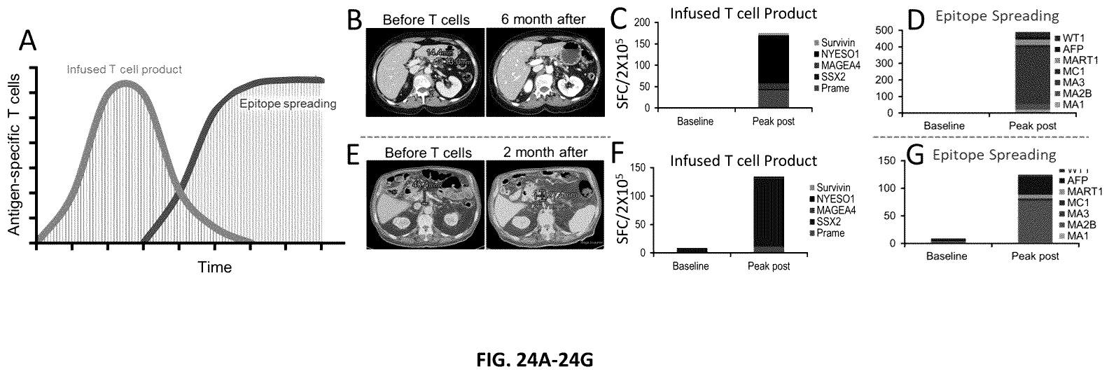

FIG. 21 shows that expression in the bone marrow of targeted tumor antigens Survivin and WT1 directly correlated with MRD. Moreover, decreases in Survivin and WT1 expression correlated with an increase in target antigen-specific T cells detected in the peripheral blood. FIG. 22 shows that expression in the bone marrow of non-target antigens (e.g., proteinase-3) is detected after product infusion, and correlates with MRD at later timepoints demonstrating epitope spreading. Further, an increase in the proportion of proteinase-3 specific T cells had an inverse correlation with proteinase-3 detected in the tumor. To understand if additional non-target antigens were decreased, RNA expression of seven non-target antigens was measured in the patient, and antigen specificity from the T cells was measured for the same non-target antigens. FIG. 23 shows that additional non-targeted tumor antigens significantly decreased after product infusion, and trend with MRD. Together, these data demonstrate epitope spreading after administration of the tumor antigen-specific T cells.

### Example 7. Epitope Spreading Following Treatment with Multiple Tumor Antigen-Specific T Cells

Epitope spreading occurs when T cells targeting specific antigens destroy them, exposing new “non-targeted” epitopes/antigens, which recruits and expands T cells specific for non-target antigens to help eliminate the tumor, thus limiting tumor escape. To further evaluate the observation of epitope spreading following administration of MultiTAA-specific T cells to patients, MultiTAA-specific T cells were tested in vitro and in vivo in different cancer types.

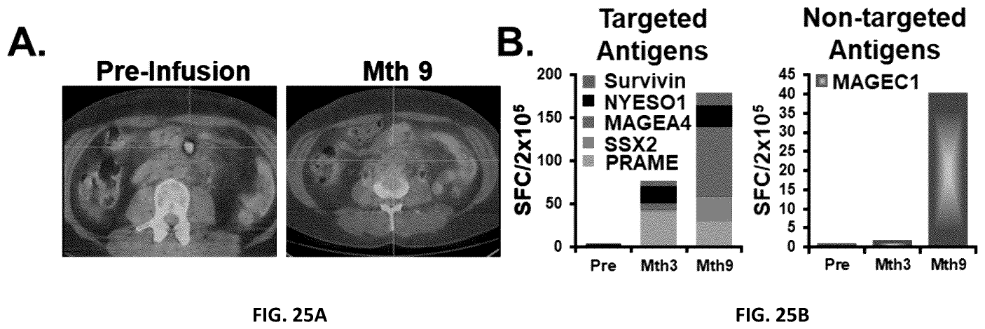

In a lymphoma clinical trial, patients were administered a MultiTAA-specific T cells and epitope spreading was observed. Specifically, patients were assessed at month 3 (Mth3) and month 9 (Mth9) post infusion of MultiTAA-specific T cells generated using the MAT101-5 method. The MAT101-5 method is identical to the MAT101-4 method except that it comprises incubation with peptides from 5 tumor-associated antigens (Survivin, NY-ESO-1, MAGEA4, SSX2, and PRAME. Nine months after the initial administration, tumor burden was reduced in patients treated with the MultiTAA-specific T cells (FIG. 25A), and antigen specificity was measured by IFN-γ ELISpot analysis. At the nine-month time point, specificity was observed for all tumor associated target antigens (FIG. 25B) as well as an increase in specificity for non-targeted antigen MAGEC1 (FIG. 25B).

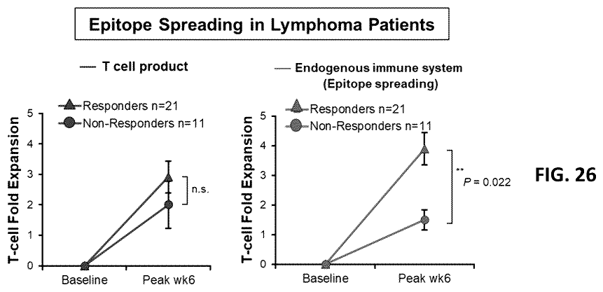

Expansion of T cells specific to “target” epitopes from the peptides used to generate the antigen-specific T cell product (left) and “non-target” epitopes indicative of epitope spreading (right) was measured in responding and non-responding lymphoma patients 6 weeks after infusion of MultiTAA-specific T cells (FIG. 26B). Significantly greater expansion of T cells specific to both “target” and “non-target” epitopes was observed in Responders compared with Non-Responders, suggesting that epitope spreading may be positively associated with response to treatment.

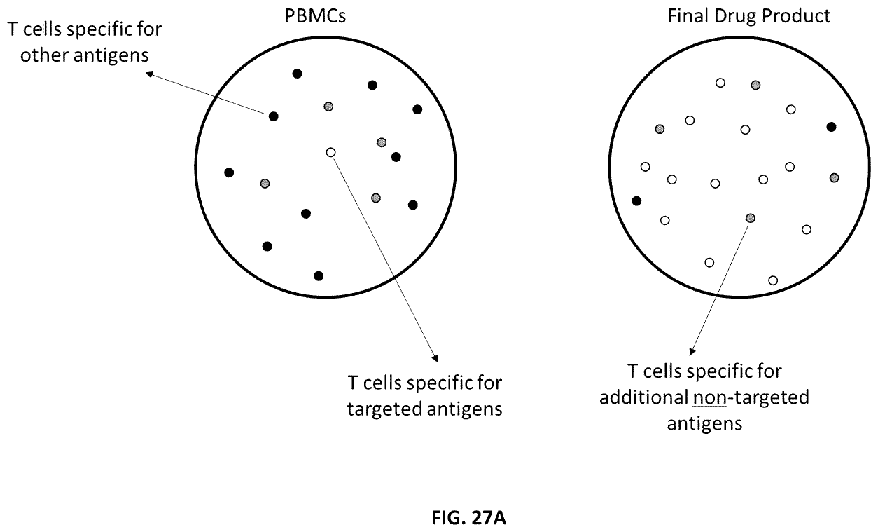

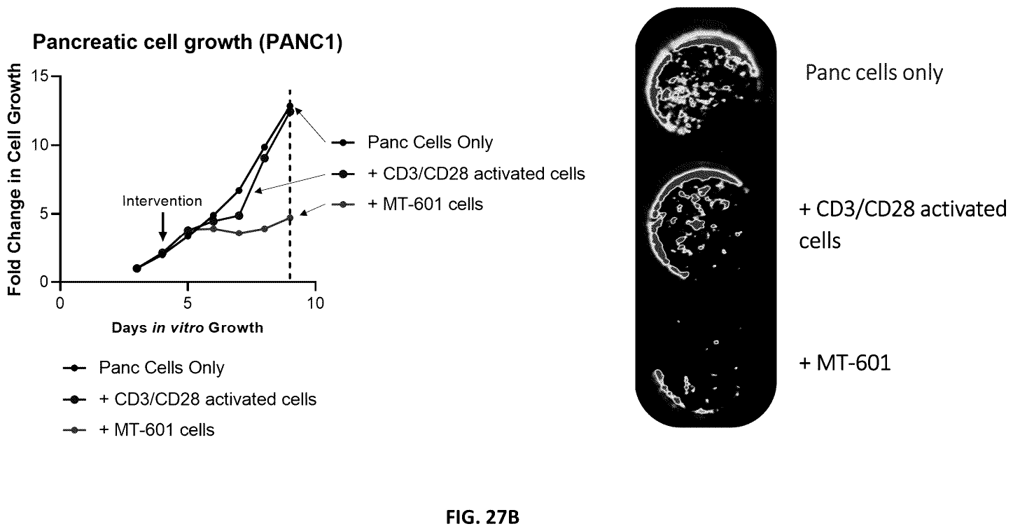

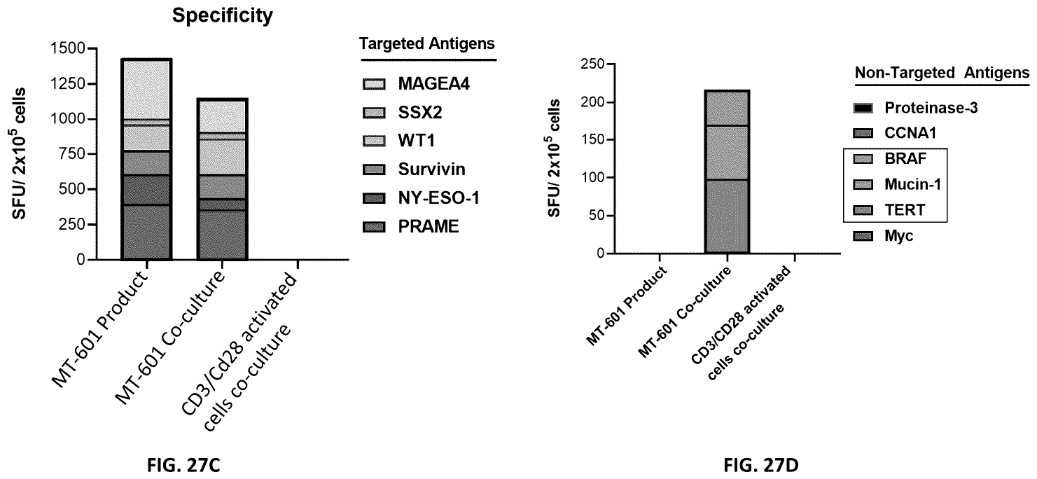

Preparation of TAA-specific cell products has generally focused on purifying and infusing a population of T cells specific for targeted antigens from the starting cell population (FIG. 27A, “PBMCs”). Surprisingly, examples presented herein show that T cells within the cell product (FIG. 27A, “Final Drug Product,” including MT-401, MT-601 or other described embodiments) have specificity for additional, non-targeted antigens. Once the targeted antigens are destroyed, additional epitopes become available and expand the T cells within the Final Drug Product specific for “non-target” antigens. Further, this “non-target” specificity is indicative of epitope spreading and may be beneficial to the overall tumor response generated by the cell product.

To evaluate whether “non-target” TAA responses within the T cell product are associated with suppression of cancer cell growth, MT-601 was further evaluated in a pancreatic cancer cell model. Using an in vitro assay target cells (i.e., pancreatic cancer cells) were co-cultured with MT-601-cells and non-specific T cells (anti-CD3/CD28 activated). When PANC1 cells were co-cultured with anti-CD3+/CD28+ activated non-specific T cells there was no suppression of cancer cell growth observed. However, co-culture with MT-601 cells resulted in a reduction in cancer cell growth (FIG. 27B). Specificity for target antigens was measured by IFN-γ ELISpot analysis, and specificity was observed for MT-601 cells alone or following co-culture with PANC1 cells, whereas there was no target antigen specificity for anti-CD3+/CD28+ activated non-specific T cells co-cultured with PANC1 cells (FIG. 27C). Due to epitope spreading, specificity for non-target antigens was found for MT-601 cells that were co-cultured with PANC1 cells, but none was observed for culture with anti-CD3+/CD28+ activated T cells (FIG. 27D). Together, this data demonstrates that T cells in the MT-601 cell product maintain specificity for target antigens and additionally increase specificity for non-target antigens, demonstrating epitope spreading.

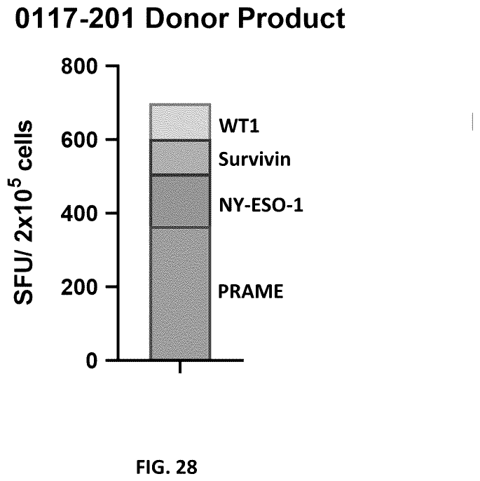

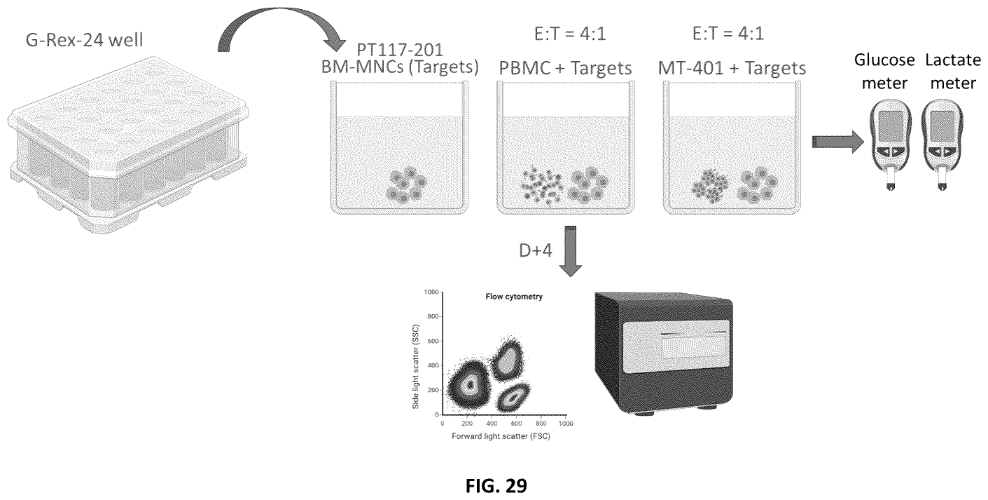

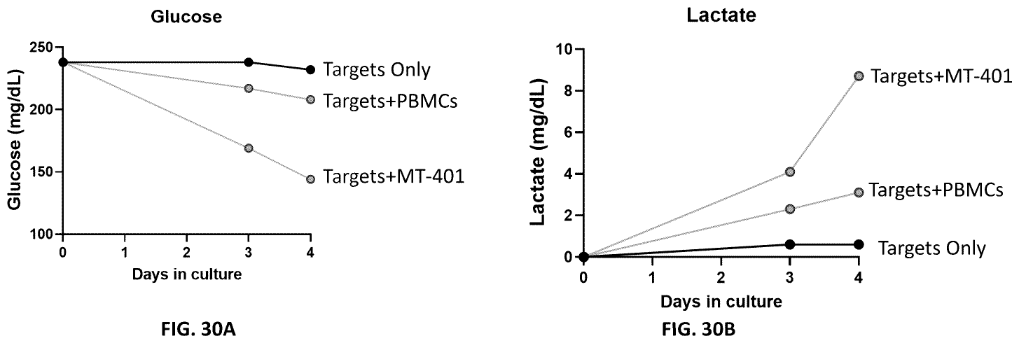

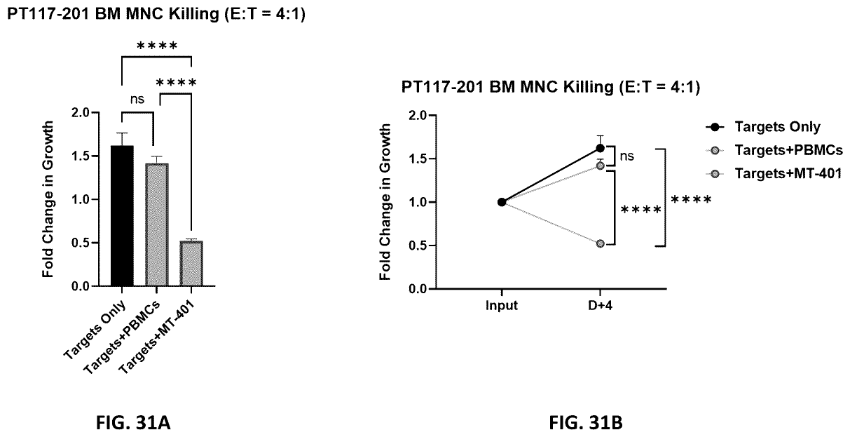

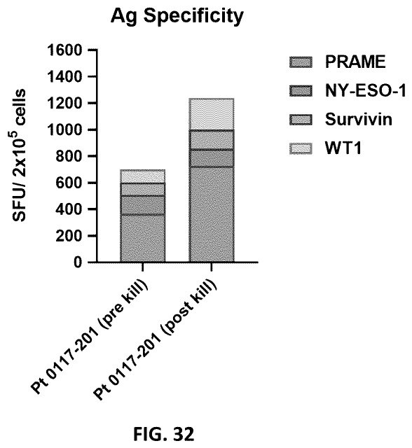

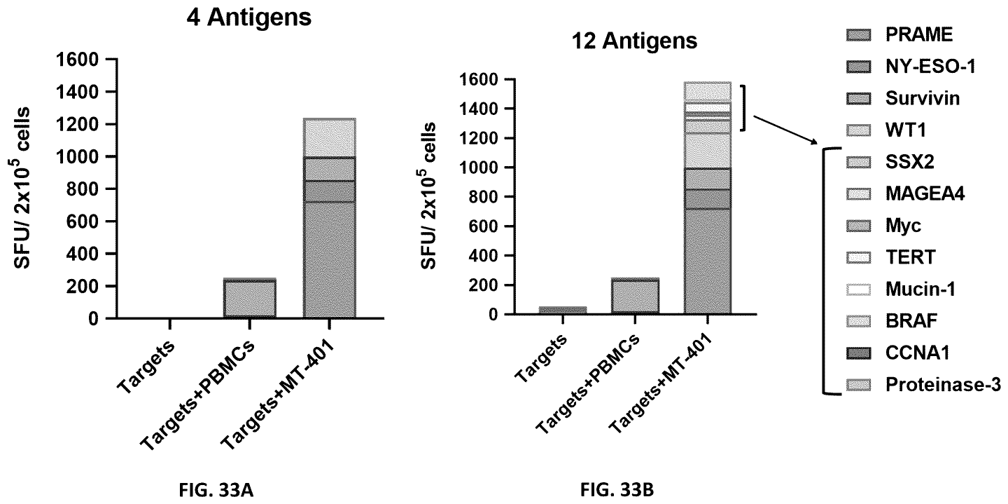

An additional MultiTAA-specific product, MT-401, was generated using the MAT110-CD56+ method with a peptide library with specificity to four antigens: PRAME, NY-ESO-1, Survivin, and WT1 (FIG. 28). MT-401 was evaluated for its anti-tumor effect on cells from a patient with Acute Myeloid Leukemia. The patient previously received Decitabine, Venetoclax and Cytarabine, then Fludarabine and Cyclophosphamide for lymphodepletion prior to a HSCT but relapsed prior to MT-401 infusion. Bone Marrow (BM) mononuclear cells were isolated from the patient at relapse and co-cultured with either control PBMCs or MT-401 cells (FIG. 29) at an effector:target (E:T) ratio of 4:1. Cells were cultured together for four days and each day glucose and lactate production was measured to monitor cell-growth. A reduction in glucose production was observed in cells cultured with MT-401, demonstrating a decrease in target cell growth (FIG. 30A). An increase in lactate production was observed in cells cultured with MT-401 correlating with T-cell proliferation (FIG. 30B). Cancer cell growth was measured at the end of the co-culture and target cancer cells cultured with MT-401 has reduced cell growth (FIGS. 31A and 31B) demonstrating improved target cell killing. The antigen specificity of the MT-401 cells was compared before and after co-culture, and specificity increased for PRAME and WT1 after culture with the cancer cells (FIG. 32). Not only was antigen specificity for the four target antigens that MT 401 was generated against maintained on the T cells (FIG. 33A), but specificity for non-target antigens was observed for MT-401 cells, as compared to PBMCs which showed no specificity for non-target antigens (FIG. 33B).

### Example 8. Donor Selection and Manufacturing Scale-Up

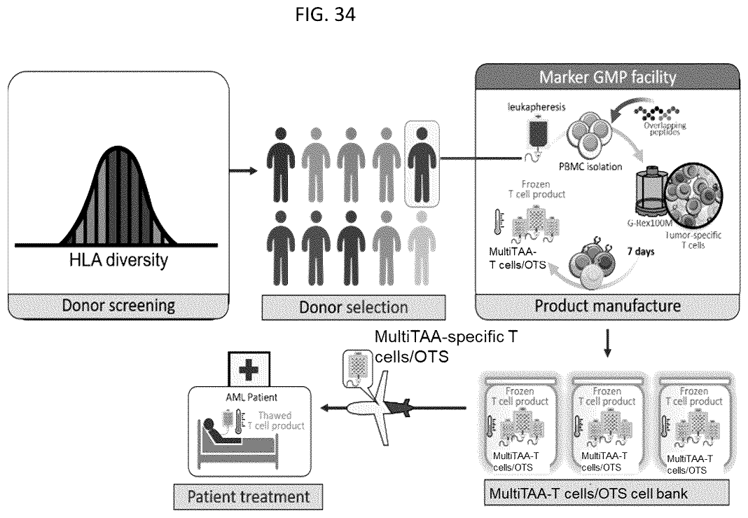

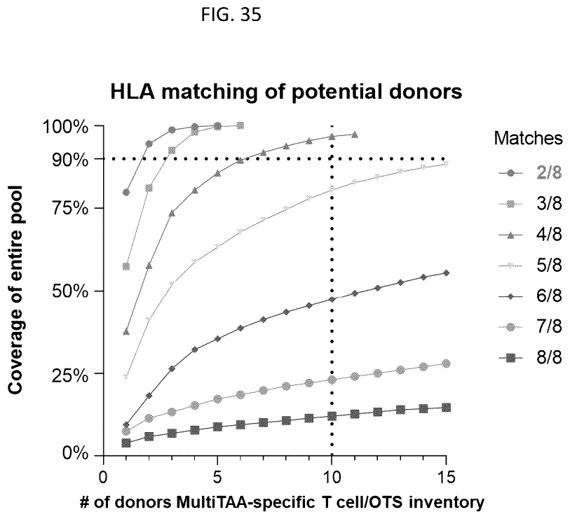

Donors are screened for HLA diversity and selected on the basis of their ability to match a broad set of HLA genotypes in a given sample population. Following selection, cells are collected by leukapheresis and prepared using the methods described herein. The antigen-specific T cells are then frozen prior to delivery and administration to a patient (FIG. 34). An off-the-shelf inventory of MultiTAA T cell products could be generated from only 10 donors that would have the ability to match over 90% of patients at 4/8 HLA alleles. Furthermore, an off-the-shelf inventory of MultiTAA T cells products generated from only 5 donors would have the ability to match almost 100% of patients at 2/8 HLA alleles (FIG. 35).

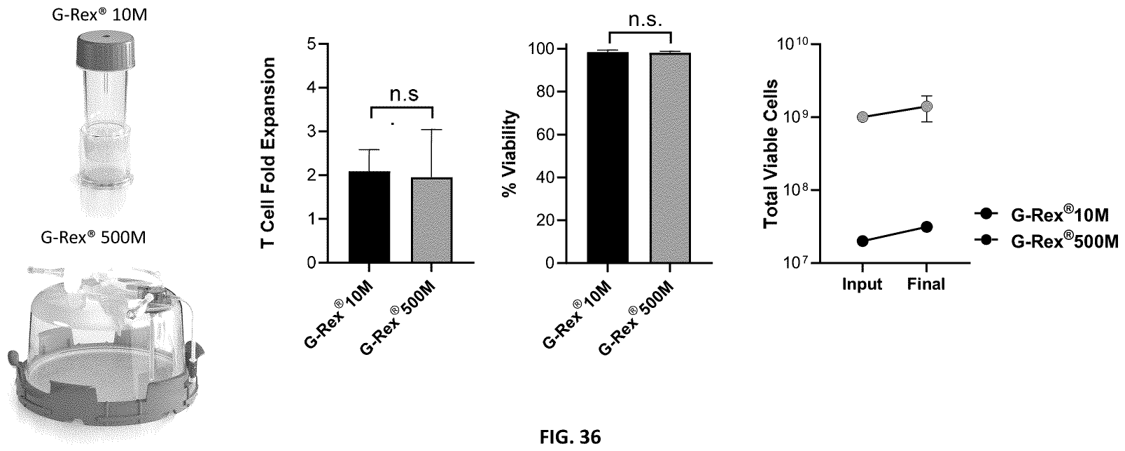

To evaluate whether the MAT110-CD56+Depl method could be scaled up for manufacturing, cells were generated in a G-Rex 10M compared to a G-Rex 500M. FIG. 36 shows that a scale-up from the G-Rex 10M to G-Rex 500M device does not alter final product cell viability or fold T cell expansion. Thus, the MAT110-CD56+Depl method provided herein is suitable for a scaled-up manufacturing process.

### Example 9. Additional Antigens Increase Specificity and Diversity of T Cells

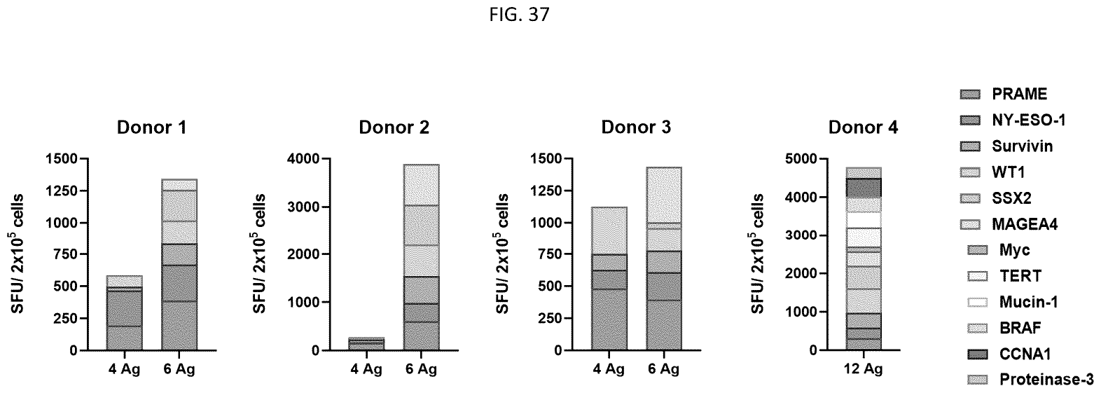

An additional study was performed to assess the effect of adding additional peptide libraries of tumor antigens to the culture mix. Three donor mononuclear cell populations were stimulated with peptide libraries from 4 tumor antigens or 6 tumor antigens according to the MAT110-CD56+Depl method provided herein. Mononuclear cells from a fourth donor were stimulated with peptide libraries from 12 tumor antigens. As shown in FIG. 37, surprisingly, the MultiTAA-specific T cell population generated using peptides from 12 tumor antigens exhibited increased diversity and specificity of T cells, with a total magnitude of response (sum of the response against each target antigen) than 4,500 SFU/2×10⁵ cells as measured by IFNγ ELISpot.

1. A method for generating tumor antigen-specific T cells comprising:
(i) placing a population of mononuclear cells obtained from a subject in a vessel comprising at least one gas permeable surface, wherein the mononuclear cells are added to the vessel at a seeding density of about 0.1×106 cells to about 9×106 cells per cm2 of the gas permeable surface to form a cell culture;
(ii) adding one or more peptides from one or more tumor antigens to the cell culture;
(iii) incubating the one or more peptides with the mononuclear cells in the vessel at about 37° C. in the absence of exogenous cytokines.

2. The method of claim 1, further comprising:
(iv) after the incubation of step (iii), adding one or more exogenous cytokines to the cell culture.

3. The method of claim 1, where exogenous cytokines are added after about 30 minutes to about 30 hours of incubation.

4. The method of claim 1, wherein the mononuclear cells are cultured in the presence of the one or more peptides and the exogenous cytokines for about 5 to about 14 days.

5. The method of claim 1, wherein each peptide of the one or more peptides of (ii) is added to the culture at a concentration of about 0.1 to about 0.5 pg/mL.

6. The method of claim 1, wherein the one or more peptides comprise one or more libraries of peptides derived from the one or more tumor antigens.

7. The method of claim 1, wherein each library of peptides comprises peptides spanning the sequence of the tumor antigen.

8. The method of claim 1, comprising adding peptides from at least 4 different tumor antigens at step (ii).

9. The method of claim 1, wherein the method further comprises depleting CD56+ cells from the population of mononuclear cells prior to (i).

10. The method of claim 9, wherein the CD56+ cells are depleted.

11. The method of claim 1, wherein the population of mononuclear cells placed in the vessel comprise no more than about 2.5% CD56+ cells.

12. The method of claim 1, wherein the one or more exogenous cytokines are selected from IL-6, IL-7, IL-15, IL-12, and any combination thereof.

13. A population of antigen-specific T cells, comprising between about 80% to about 99% CD3+ T cells, <1% CD14+ monocytes, <10% CD19+ B cells, and <10% CD3-CD56+NK cells, wherein the population of antigen-specific T cells has activity against one or more peptides of one or more antigens that the population of T cells has not been previously incubated with.

14. The population of claim 13, wherein the magnitude of specificity against the at least one peptide of at least one antigen that the population of T cells has not been previously incubated with is, cumulatively, at least about 100 SFU/2×105 cells as measured by IFN-y ELISpot.

15. The population of claim 14, wherein the magnitude of specificity against at least one antigen that the population has not been previously exposed to or incubated with is at least about 30 SFU/2×105 cells as measured by IFN-y ELISpot.

16. The population of claim 13 wherein the population has previously been incubated with one or more peptides from one or more tumor antigens in the absence of exogenous cytokines.

17. The population of claim 13, wherein the magnitude of specificity of the antigen-specific T cells for the one or more tumor antigens is at least about 500 SFU/2×105 cells as measured by IFN-y ELISpot.

18. The population of claim 13, wherein the antigen-specific T cells comprise at least about 20% central memory T cells (TCM).

19. The population of claim 13, wherein the antigen-specific T cells comprise about 40% effector memory T cells (TEM).

20. The population of claim 13, wherein the antigen-specific T cells comprise no more than 10% terminally differentiated effector memory cells expressing CD45RA (TEMRA).

21. The population of claim 13, wherein the antigen-specific T cells comprise at least about 20% naive T cells.

22. The population of claim 13, wherein the antigen-specific T cells exhibit activity against at least 75% of the tumor antigens represented by the one or more peptides.

23. The population of claim 13, wherein the antigen-specific T cells exhibit activity against each tumor antigen represented by the one or more peptides.

24. The population of claim 23, wherein the activity against the antigens is detected by IFNγ production.

25. The population of claim 24, wherein the IFNy production is determined by IFNy ELISpot assay.

26. The population of claim 13, wherein an increased number of tumor antigens represented by the one or more peptides results in (a) an increase in specificity of the antigen specific T cells and/or (b) an increase in specificity of the antigen-specific T cells for the tumor antigens.

27. The population of claim 13, wherein T cells within said population exhibit activity against one or more peptides of one or more antigens that the population has not been previously incubated with.
